(19) United States
(12) Patent Application Publication
(10) Pub. No.: US 2015/0080930 A1
(43) Pub. Date: Mar. 19, 2015

(54) PUNCTURE APPARATUS AND PUNCTURING METHOD

(71) Applicant: TERUMO KABUSHIKI KAISHA, Shibuya-ku (JP)

(72) Inventors: Masakatsu Kawaura, Sunnyvale, CA (US); Nao Yokoi, Sunnyvale, CA (US)

(73) Assignee: TERUMO KABUSHIKI KAISHA, Tokyo (JP)

(21) Appl. No.: 14/489,087

(22) Filed: Sep. 17, 2014

(30) Foreign Application Priority Data
Sep. 17, 2013 (JP) 2013-192439

(51) Int. Cl. A61B 17/34

(52) U.S. Cl. CPC A61B 17/3417
USPC 606/185

(57) ABSTRACT

A puncture apparatus and a puncturing method are disclosed by which a biological tissue is punctured. The puncture apparatus can include an elongated curveable puncture needle which punctures a biological tissue, and a curvature-changing unit which changes curvature of the puncture needle in a process of puncturing the biological tissue by the puncture needle. The puncturing method can include puncturing in such a manner that curvature of the puncture hole varies along a longitudinal direction of the puncture hole, in a process of puncturing the biological tissue.

### CROSS-REFERENCES TO RELATED APPLICATIONS

This application claims priority to Japanese Application No. 2013-192439 filed on Sep. 17, 2013, the entire content of which is incorporated herein by reference.

### TECHNICAL FIELD

The present disclosure relates to a puncture apparatus and a puncturing method.

### BACKGROUND DISCUSSION

In a patient suffering from urinary incontinence, for example, stress urinary incontinence, urine leakage (involuntary urination) occurs due to an abnormal pressure exerted during a normal exercise or by laughing, coughing, sneezing or the like. This can be attributable, for example, to loosening of a pelvic floor muscle, which is a muscle supporting a urethra, caused by childbirth or the like.

For treatment of urinary incontinence, surgical therapy is effective. For example, a tape-shaped implant called “sling” is placed indwelling in the body to support the urethra by the sling (for example, U.S. Pat. No. 6,911,003). In order to put a sling indwelling in the body, an operator can incise a vagina with a surgical knife, dissects a biological tissue (living body tissue) between the urethra and the vagina, and provides communication between the exfoliated biological tissue site and an exterior through an obturator foramen by using a puncture needle or the like which is curved in an arcuate shape (this operation will hereinafter be referred to as “puncturing operation”). Then, in such a state, the sling is placed indwelling in the body.

In the case where the puncturing operation is carried out using a puncture needle having a comparatively large radius of curvature there can be issues. For example, although a puncture to a good depth from a body surface can be achieved by this operation, there can be a risk of erroneously puncturing a blood vessel such as common iliac artery and veins, femoral artery and veins, obturator artery and veins, etc. or nerve such as obturator nerve. For example, where a puncturing operation using a puncture needle with a comparatively small radius of curvature is applied to a comparatively large patient where a distance from a body surface to a tissue between a urethral lumen and a vaginal lumen (vaginal cavity) is long, the puncture needle may fail to satisfactorily reach a target site, depending on the length of the puncture needle.

### SUMMARY

In accordance with an exemplary embodiment, a puncture apparatus and a puncturing method are disclosed by which a biological tissue can be punctured relatively accurately and satisfactorily.

In accordance with an exemplary embodiment, a puncture apparatus is disclosed, which can include a puncture needle which punctures a biological tissue, the puncture needle being elongated and able to be curved; and a curvature-changing unit which changes curvature of the puncture needle in a process of puncturing the biological tissue by the puncture needle.

In accordance with an exemplary embodiment, the curvature-changing unit can be configured to cause the puncture needle to sequentially perform a first puncture and a second puncture greater than the first puncture in the curvature of the puncture needle.

In accordance with an exemplary embodiment, the puncture apparatus is for use in forming the biological tissue with a puncture hole in which an implant is to be placed indwelling between a urethral lumen and a vaginal lumen, prior to the placement; and the first puncture is performed during when a needle tip of the puncture needle is moved from a living body surface to a point of immediately before reaching an obturator foramen on one side, and the second puncture is performed during when the needle tip of the puncture needle is moved from the point of immediately before reaching the obturator foramen on the one side to cross a region between the urethral lumen and the vaginal lumen and is moved past an obturator foramen on other side.

In accordance with an exemplary embodiment, a restriction unit is disclosed, which performs the first puncture; and a curved portion which performs the second puncture, the curved portion so configured as to be insertable in the restriction unit, wherein the restriction unit restricts curvature of the curved portion.

In accordance with an exemplary embodiment, the curved portion is curved in an arcuate shape in a natural state.

In accordance with an exemplary embodiment, an operating unit is disclosed which deforms the curved portion into an arcuate state.

In accordance with an exemplary embodiment, the curved portion is so configured that its rotation about an axis along a direction of puncture is restricted by insertion of the curved portion in the restriction unit.

In accordance with an exemplary embodiment, a method of puncturing a biological tissue to form the biological tissue with a bow-like puncture hole is disclosed, the method including: puncturing in such a manner that curvature of the puncture hole varies along a longitudinal direction of the puncture hole, in a process of puncturing the biological tissue.

In accordance with an exemplary embodiment, in the process of puncturing a biological tissue to form a puncture hole, the curvature of the puncture needle can be appropriately changed according to the depth of puncture. In addition, attendant on the variation in the curvature of the puncture needle, the puncture hole thus formed is also varied in curvature along the longitudinal direction thereof. Thus, erroneous punctures of common iliac artery or issues in which the puncture needle cannot reach a target site can be prevented. Consequently, a biological tissue can be puncture relatively accurately and satisfactorily.

### BRIEF DESCRIPTION OF THE DRAWINGS

FIG. 1 is a perspective view showing a first exemplary embodiment of a puncture apparatus according to the present disclosure.

FIG. 2 is a side view of the puncture apparatus shown in FIG. 1.

FIG. 3 is a plan view showing a fixing portion of a frame possessed by the puncture apparatus shown in FIG. 1.

FIG. 4 is a side view of an insertion instrument possessed by the puncture apparatus shown in FIG. 1.

FIGS. 5A and 5B illustrate a positional relationship between a puncture member and obturator foramens (pelvis), wherein FIG. 5A is a side view and FIG. 5B is a front view.

FIG. 6 is a partial magnified view of a vaginal insertion member possessed by the insertion instrument shown in FIG. 4.

FIG. 7A is a sectional view showing an example of a shape of a vaginal wall, and FIG. 7B is a sectional view showing a state in which a vaginal insertion portion has been inserted in an inside of the vagina shown in FIG. 7A.

FIGS. 8A and 8B illustrate a puncture member possessed by the puncture apparatus shown in FIG. 1, wherein FIG. 8A is a perspective view and FIG. 8B is a sectional view.

FIGS. 9A and 9B are views illustrating an operation procedure of the puncture apparatus shown in FIG. 1.

FIG. 10 is a view illustrating the operation procedure of the puncture apparatus shown in FIG. 1.

FIG. 11 is a side view illustrating a relationship between the puncture apparatus and the pelvis in the state shown in FIG. 10.

FIG. 12 is a view (as viewed from a patient's leg side) illustrating the operation procedure of the puncture apparatus shown in FIG. 1.

FIG. 13 is a view (as viewed from the patient's leg side) illustrating the operation procedure of the puncture apparatus shown in FIG. 1.

FIG. 14 is a view (as viewed from the patient's leg side) illustrating the operation procedure of the puncture apparatus shown in FIG. 1.

FIG. 15 is a view (as viewed from the patient's leg side) illustrating the operation procedure of the puncture apparatus shown in FIG. 1.

FIG. 16 is a view (as viewed from the patient's leg side) illustrating the operation procedure of the puncture apparatus shown in FIG. 1.

FIG. 17 is a view (as viewed from the patient's leg side) illustrating the operation procedure of the puncture apparatus shown in FIG. 1.

FIG. 18 is a sectional view taken along line 18-18 in FIG. 14.

FIG. 19 is a sectional view taken along line 19-19 in FIG. 16.

FIG. 20 is a sectional view taken along line 20-20 in FIG. 17.

FIG. 21 is a view illustrating an operation procedure of the puncture apparatus in accordance with a second exemplary embodiment of the present disclosure.

FIG. 22 is a view illustrating the operation procedure of the puncture apparatus according to the present disclosure.

FIG. 23 is a side view showing a third exemplary embodiment of the puncture apparatus according to the present disclosure.

FIG. 24 is a side view showing a fourth exemplary embodiment of the puncture apparatus according to the present disclosure.

FIG. 25 is a perspective view showing a fifth exemplary embodiment of the puncture apparatus according to the present disclosure.

FIG. 26 is a plan view showing a puncture member possessed by the puncture apparatus shown in FIG. 25.

FIG. 27 is a perspective view showing the puncture member possessed by the puncture apparatus shown in FIG. 25.

FIG. 28 is a sectional view of the puncture member shown in FIG. 27.

FIGS. 29A to 29C illustrate a state-maintaining mechanism possessed by the puncture member shown in FIG. 27, wherein FIG. 29A is a top plan view and FIGS. 29B and 29C are sectional views.

FIGS. 30A to 30C are partial magnified views showing the state-maintaining mechanism possessed by the puncture member shown in FIG. 26, wherein FIGS. 30A and 30B are plan views showing modifications, respectively, and FIG. 30C is a plan view showing this exemplary embodiment.

FIG. 31 is a view (as viewed from the patient's leg side) illustrating an operation procedure of the puncture apparatus shown in FIG. 25.

FIG. 32 is a view (as viewed from the patient's leg side) illustrating the operation procedure of the puncture apparatus shown in FIG. 25.

FIG. 33 is a view (as viewed from the patient's leg side) illustrating the operation procedure of the puncture apparatus shown in FIG. 25.

FIG. 34 is a view (as viewed from the patient's leg side) illustrating the operation procedure of the puncture apparatus shown in FIG. 25.

FIG. 35 is a view (as viewed from the patient's leg side) illustrating the operation procedure of the puncture apparatus shown in FIG. 25.

FIG. 36 is a view (as viewed from the patient's leg side) illustrating the operation procedure of the puncture apparatus shown in FIG. 25.

FIGS. 37A to 37D illustrate operating states of a support unit possessed by the puncture apparatus shown in FIG. 25.

FIGS. 38A and 38B illustrate a support unit possessed by the puncture apparatus in accordance with a sixth exemplary embodiment of the present disclosure, wherein FIG. 38A is a plan view and FIG. 38B is a cross-sectional view.

FIG. 39 is a view (as viewed from the patient's leg side) illustrating an operation procedure of the puncture apparatus in accordance with a seventh exemplary embodiment of the present disclosure.

FIG. 40 is a view (as viewed from the patient's leg side) illustrating the operation procedure of the puncture apparatus according to the present disclosure.

FIG. 41 is a view (as viewed from the patient's leg side) illustrating the operation procedure of the puncture apparatus according to the present disclosure.

FIG. 42 is a view (as viewed from the patient's leg side) illustrating the operation procedure of the puncture apparatus according to the present disclosure.

FIG. 43 is a view (as viewed from the patient's leg side) illustrating the operation procedure of the puncture apparatus according to the present disclosure.

FIG. 44 is a view (as viewed from the patient's leg side) illustrating the operation procedure of the puncture apparatus according to the present disclosure.

FIG. 45 is a view (as viewed from the patient's leg side) illustrating the operation procedure of the puncture apparatus according to the present disclosure.

FIG. 46 is a view (as viewed from the patient's leg side) illustrating the operation procedure of the puncture apparatus according to the present disclosure.

FIGS. 47A to 47E illustrate operating states of a support unit possessed by the puncture apparatus according to the present disclosure.

FIGS. 48A and 48B are sectional views showing an eighth exemplary embodiment of the puncture apparatus according to the present disclosure.

FIG. 49 is a perspective view showing a puncture member possessed by the puncture apparatus in accordance with a ninth exemplary embodiment of the present disclosure.

FIG. 50 is a sectional view showing a modification of the puncture member shown in FIG. 49.

### DETAILED DESCRIPTION

A puncture apparatus and a puncturing method according to the present disclosure will be described in detail below, referring to exemplary embodiments illustrated in the accompanying drawings.

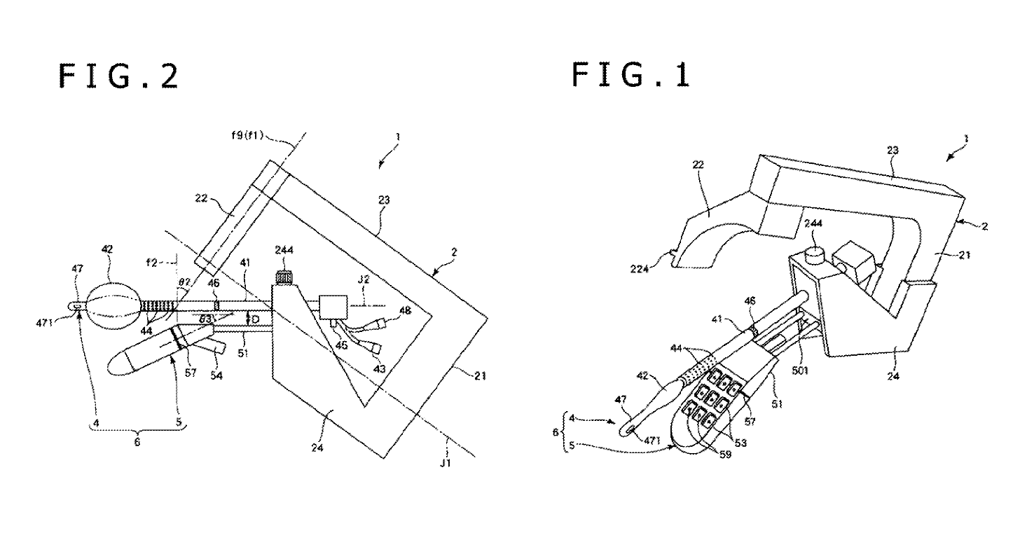

FIGS. 1-20 illustrate a first exemplary embodiment of a puncture apparatus according to the present disclosure. In the following disclosure, for convenience of description, a left side in FIG. 2 will be referred to as “distal (side),” a right side as “proximal (side),” an upper side as “upper (side),” and a lower side as “lower (side).” In addition, FIG. 2 shows the puncture apparatus in a state of being yet to be used, which state will hereinafter be referred to also as “initial state,” for convenience of description. Further, a state wherein the puncture apparatus (insertion instrument) shown in FIG. 2 is mounted on a patient will be referred to also as “mounted state.”

In accordance with an exemplary embodiment, a puncture apparatus 1, which can be used for treatment of female urinary incontinence is disclosed, for example, to be used in a process in which a biological tissue-supporting implant for treatment of urinary incontinence is implanted into a living body.

As shown in FIGS. 1 and 13, the puncture apparatus 1 can include a frame (support unit) 2, a puncture member 3A, a urethral insertion member 4, and a vaginal insertion member 5. The puncture member 3A, the urethral insertion member 4, and the vaginal insertion member 5 are supported on the frame 2. In the puncture apparatus 1, besides, the urethral insertion member 4 and the vaginal insertion member 5 constitute an insertion instrument 6.

As shown in FIGS. 1 and 2, the frame 2 can include a grasping portion 21 to be grasped in the mounted state, a guide portion (holding portion) 22 guiding the puncture member 3A, an interlock portion 23 interlocking the grasping portion 21 and the guide portion 22 to each other, and a fixing portion 24 to which the insertion instrument 6 is fixed.

The grasping portion 21 is located on a proximal end in the puncture apparatus 1, and extends in a direction substantially orthogonal to an axis J1. The “axis J1” refers to an axis, which serves as a center (axis) of turn when the puncture member 3A is turned.

The guide portion 22 is located on a distal end in the puncture apparatus 1, and is disposed opposite to the grasping portion 21. The guide portion 22 can be arch-shaped, and can be provided with an engaging portion 224 at an outer circumferential portion of a lower end of the guide portion 22. The engaging portion 224 can be composed essentially of a projection which projects outward.

The interlock portion 23 can interlock the grasping portion 21 and the guide portion 22 to each other. In addition, the interlock portion 23 can be in a form of a bar extending substantially parallel to the axis J1. The interlock portion 23 can also function as a grasping portion, like the grasping portion 21, to allow an operator to use the puncture apparatus 1 by grasping the interlock portion 23.

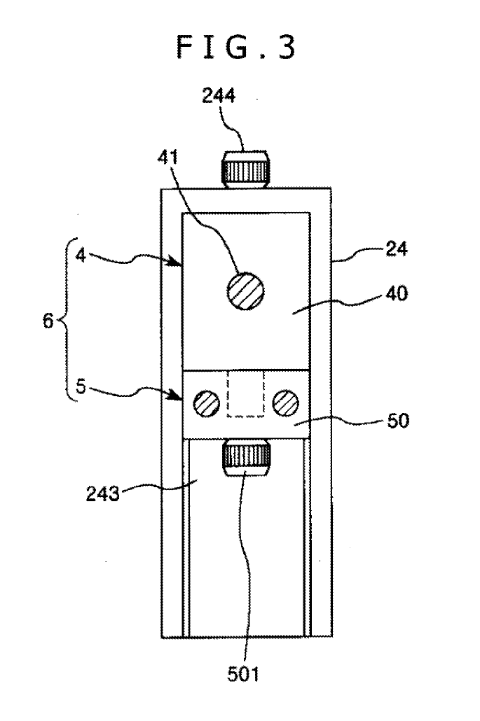

The fixing portion 24 is disposed opposite to the interlock portion 23, with the axis J1 interposed therebetween. As shown in FIG. 3, the fixing portion 24 is provided with a recess 243 in which to fit a support part 60 (described later) of the insertion instrument 6, and a male screw 244. With the support part 60 fitted in the recess 243 and with the male screw 244 fastened into a female screw (not shown) in the support part 60, the insertion instrument 6 can be fixed to the fixing portion 24.

The puncture member 3A is a member puncturing a living body (biological tissue). As shown in FIGS. 13 to 16, the puncture member 3A can include a puncture needle 11, a sheath (medical tube) 30, a connection member 12 and a restriction member 13, which constitute an assembly.

The puncture needle 11 is configured by use of an elongated wire material, and can be elastic, which help permits the puncture needle 11 to be elastically deformed into a curved shape. The puncture needle 11 can be rectilinear in shape in a natural state with no external force exerted on the puncture needle 11.

The puncture needle 11 may be formed of a metallic material. The metallic material is not specifically restricted, and examples of the metallic material can include superelastic alloys such as Ni—Ti alloys, Ni—Al alloys and Cu—Zn alloys.

A distal portion of the puncture needle 11 can be in a tapered shape, with its outside diameter decreasing gradually along the distal direction, which can result in that the puncture needle 11 is formed at its distal end with a sharp needle tip 111. This needle tip 111 is a part, which mainly bears puncturing of a living body.

In accordance with an exemplary embodiment, an outer circumferential portion of the puncture needle 11 has preferably been subjected to a friction-lowering treatment by application of lubricant, for example. Such a treatment permits an operation of inserting the puncture needle 11 into the sheath 30 to be carried out relatively easily.

The sheath 30 can include a tube-shaped main body 31, and a mounting portion 37 provided at a proximal portion of the main body 31.

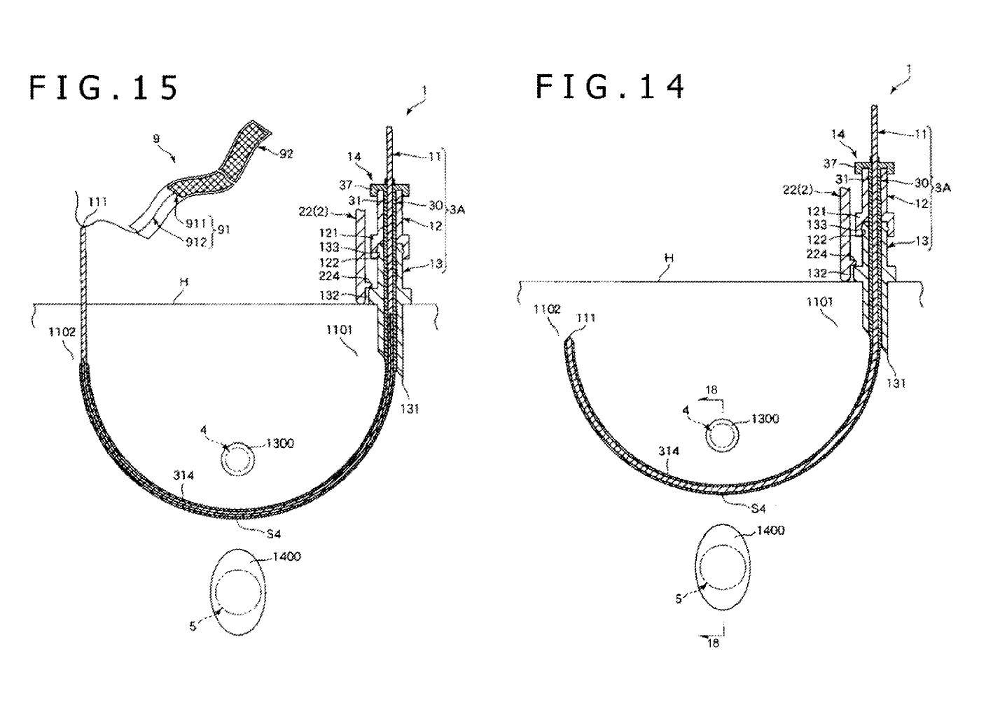

The main body 31 is configured by use of an elongate tube, which has an opening at a distal end and a proximal end of the elongate tube. In accordance with an exemplary embodiment, the main body 31 can have an internal space in which the puncture needle 11 and an implant 9 can be inserted. As shown in FIG. 14, the main body 31, with the puncture needle 11 inserted therein (inserted state), can pierce (puncture) a living body together with the puncture needle 11. An edge portion of a distal opening portion of the main body 31 is preferably in a tapered shape, with its outside diameter decreasing gradually along the distal direction, which helps ensure that in the inserted state, the edge portion of the main body 31 and the distal portion of the puncture needle 11 form a continuous tapered surface, so that puncturing of a biological tissue is facilitated. The overall lengths of the puncture needle 11 and the implant 9 can be sufficiently greater than the overall length of the main body 31.

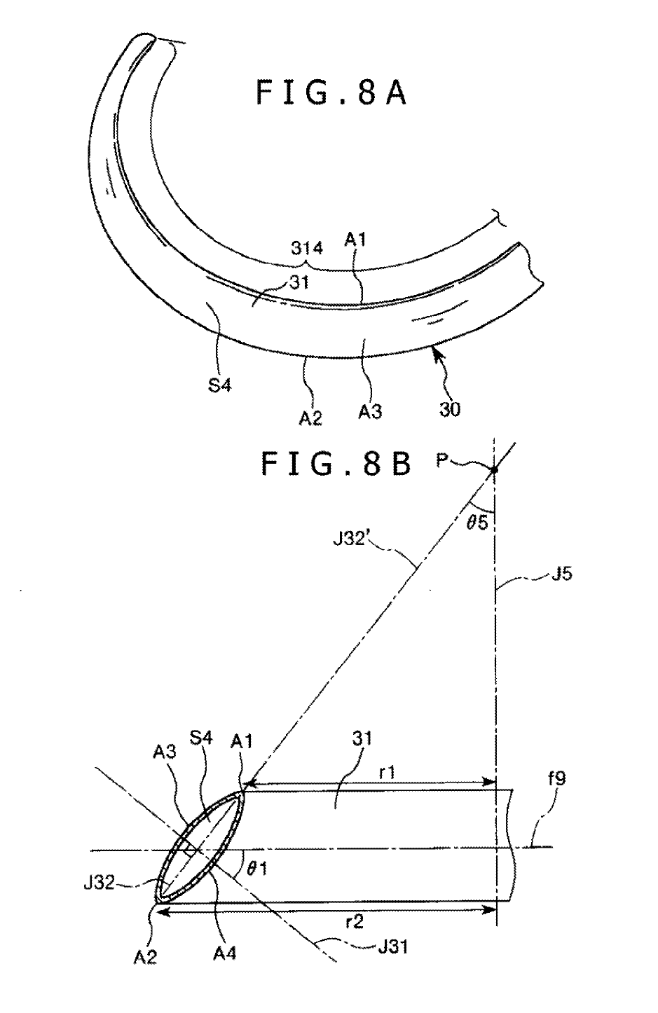

As shown in FIG. 8A, the main body 31 has a curved portion 314 ranging at least to a predetermined distance from the distal end of the main body 31. The curved portion 314 can be a portion which has been so shaped that it will be in an arc-shaped (arcuate) curved form in a natural state with no external force exerted on the curved portion 314.

In accordance with an exemplary embodiment, a center angle of the curved portion 314 is not limited, and is set, as necessary, according to various conditions. For example, as will be described later, the center angle can be set that the curved portion 314 can enter a patient body via an inguinal region on one side of the patient together with the puncture needle 11 and can be moved past a region between a urethra 1300 and a vagina 1400, to reach a vicinity of an inguinal region on the other side. For example, the center angle can be in the range of 150 to 270 degrees, more preferably 170 to 250 degrees, and further preferably 190 to 230 degrees.

In addition, the main body 31 can be configured that at least its curved portion 314 is elastic. As shown in FIG. 13, the curved portion 314 is restricted into a rectilinear shape by the restriction member 13.

As shown in FIG. 8B, the main body 31 is flat-shaped in cross section. For example, the cross-sectional shape of the curved portion 314 at a central portion S4 in the longitudinal direction of the main body 31 is a flat shape which includes a minor axis J31 and a major axis J32. As will be described later, an implant main body 91 is disposed inside the main body 31, in place of the puncture needle 11. For example, with the main body 31 flat-shaped, a posture of the implant main body 91 inside the main body 31 can be controlled.

In accordance with an exemplary embodiment, a width (a length in a direction of the major axis J32) of an internal space of the main body 31 can be designed to be approximately equal to a width of a main body portion 911 (described later) of the implant main body 91 (see FIG. 19), which can help ensure that even when the implant main body 91 is moved, a frictional resistance between the implant main body 91 and the internal space of the main body 31 is lowered, and no relatively unnecessary force can be applied to the implant main body 91, so that the main body portion 911 can be disposed in a sufficiently expanded (spread) state in the inside of the main body 31. The width (the length in the direction of the major axis J32) of the internal space of the main body 31 may be shorter than the width of the main body portion 911, which can help ensure that the width of the main body 31 is set smaller, so that a less invasive puncture member 3A can be realized.

In accordance with an exemplary embodiment, the flat shape of the main body 31 is not specifically restricted. Examples of the flat shape applicable here include ellipses, sectionally convexed lens-like shapes, rounded-cornered rhombuses, rounded-cornered rectangles (flat shapes), and spindle-like shapes enlarged (enlarged in diameter) at a central portion as compared with both end portions of the main body 31.

Hereinafter, for convenience of description, as shown in FIG. 8B, an end portion located on an inner side (one end portion) in the direction of the major axis J32 will be referred to also as “inner circumferential portion A1,” an end portion located on an outer side (other end portion) will be referred to also as “outer circumferential portion A2,” a surface oriented upward will be referred to also as “front surface A3,” and a surface oriented downward will be referred to also as “back surface A4.”

When a plane containing both the center of the arc of the central portion S4 and the center of the cross-sectional shape relative to the longitudinal direction of the main body 31 (a plane containing the center axis of the main body 31) is referred to as plane f9 and an angle formed between the plane f9 and the minor axis J31 at the central portion S4 is referred to as inclination angle θ1, the inclination angle θ1 is preferably an acute angle. With the inclination angle θ1 set to be an acute angle, the implant 9 (described later) can be disposed substantially in parallel to the urethra (urethral lumen) 1300, whereby the urethra 1300 can be effectively supported.

The inclination angle θ1 is not limited, insofar as it is an acute angle. In accordance with an exemplary embodiment, the inclination angle θ1 is preferably about 20 to 60 degrees, more preferably about 30 to 45 degrees, and further preferably about 35 to 40 degrees, which can help ensure that the above-mentioned effect is further enhanced.

In accordance with an exemplary embodiment, it can be preferable for the inclination angle θ1 to satisfy the above-mentioned numerical range throughout a whole region in an extending direction of the curved portion 314. However, the above-mentioned effect can be displayed if only the inclination angle θ1 satisfies the above-mentioned numerical range at least in the central portion S4 in the extending direction of the curved portion 314. In accordance with an exemplary embodiment, the above-mentioned “central portion S4” refers to a region that includes a part located between the urethra 1300 and the vagina (vaginal lumen) 1400, at least, in a state wherein the puncture member 3A is puncturing a living body (a state wherein the main body 31 is disposed in a living body).

The configuration of the main body 31 can also be expressed as shown in FIG. 8B, where the main body 31 is so formed that the major axis J32 is inclined against a center axis J5 of the arc and that the center axis J5 of the arc and an extension line J32′ of the major axis J32 have an intersection P. For example, an angle θ5 formed between the center axis J5 and the extension line J32′ can be equal to the inclination angle θ1. For example, as shown in FIG. 8B, the main body 31 can have the inner circumferential portion A1 located at an inner circumferential edge (in plan view as viewed from the direction of the center axis J5 of the main body 31) and can have a minimum radius of curvature, r1, and also has the outer circumferential portion A2 located at an outer circumferential edge (in the plan view) and can have a maximum radius of curvature, r2, and that the inner circumferential portion A1 and the outer circumferential portion A2 are located to be spaced (deviated) from each other along the direction of the center axis J5.

As disclosed above, the main body 31 is flat-shaped and is insusceptible to collapse in the longitudinal direction of the main body 31, so that the spacing between the inner circumferential portion A1 and the outer circumferential portion A2 will hardly be varied. In addition, the inner circumferential portion A1 and the outer circumferential portion A2 can be greater in curvature and are less susceptible to deformation, as compared with the front surface A3 and the back surface A4.

The mounting portion 37 can be provided at an outer circumferential portion of the proximal end (upper end portion) of the main body 31. The mounting portion 37 is formed along the circumferential direction of the main body 31 so as to project in a ring shape, and can be fitted to a proximal portion of the connection member 12 from the outer circumference side of the latter (see FIGS. 14 to 16), which can help ensure that the sheath 30 is detachably mounted to the connection member 12 through the mounting portion 37.

The sheath 30 can be formed of a material that is elastic and that maintains the shape and the internal space of the sheath 30 in a state wherein the sheath 30 is inserted in a body. Examples of the material applicable here include various resin materials such as polyethylene, polyimides, polyamides, polyester elastomers, and polypropylene.

The sheath 30 can be light-transmitting so that the inside of the sheath 30 is externally visible, which can help ensure, for example, that the conditions of the puncture needle 11 or the implant 9 inserted in the sheath 30 can be visually checked.

In addition, an outer circumferential portion of the sheath 30 has preferably been subjected to a friction-lowering treatment by application of lubricant, for example, which permits an operation of inserting the sheath 30 into the connection member 12 and the restriction member 13 to be carried out easily.

As shown in FIG. 13, the connection member 12 can be composed essentially of a tubular body which is higher than the sheath 30 in rigidity. The tubular body has a rectilinear center axis. In addition, an inside diameter of the connection member 12 can be slightly greater than the outside diameter of the main body 31 of the sheath 30, which can help ensure that as shown in FIG. 13, when the curved portion 314 of the sheath 30 is inserted in the connection member 12, the curved portion 314 is restricted into a rectilinear shape by the connection member 12, so that the curved portion 314 can be easily introduced as it is into the restriction member 13.

In accordance with an exemplary embodiment, the connection member 12 can have at its distal end portion (lower end portion) a diametrically enlarged portion 121 enlarged in inside diameter. The diametrically enlarged portion 121 can be fitted over a proximal portion of the restriction member 13 from an outer circumference side of the latter (see FIGS. 13 to 16), which can help ensure that the connection member 12 is detachably connected to the restriction member 13 through the diametrically enlarged portion 121.

An inner circumferential portion of the diametrically enlarged portion 121 can be formed with a groove 122 along its circumferential direction. In this groove 122 is inserted a projection 133 of the restriction member 13, in a state wherein the connection member 12 is connected to the restriction member 13, which can help ensure that the diametrically enlarged portion 121 and the restriction member 13 engage with each other, and, by this engagement as well as the above-mentioned fitting, a connected state of the connection member 12 and the restriction member 13 is maintained assuredly.

The material constituting the connection member 12 is not specifically restricted. Examples of the material applicable here include not only the same materials as those for the sheath 30 but also various metallic materials such as stainless steels, aluminum, aluminum alloys, titanium, and titanium alloys.

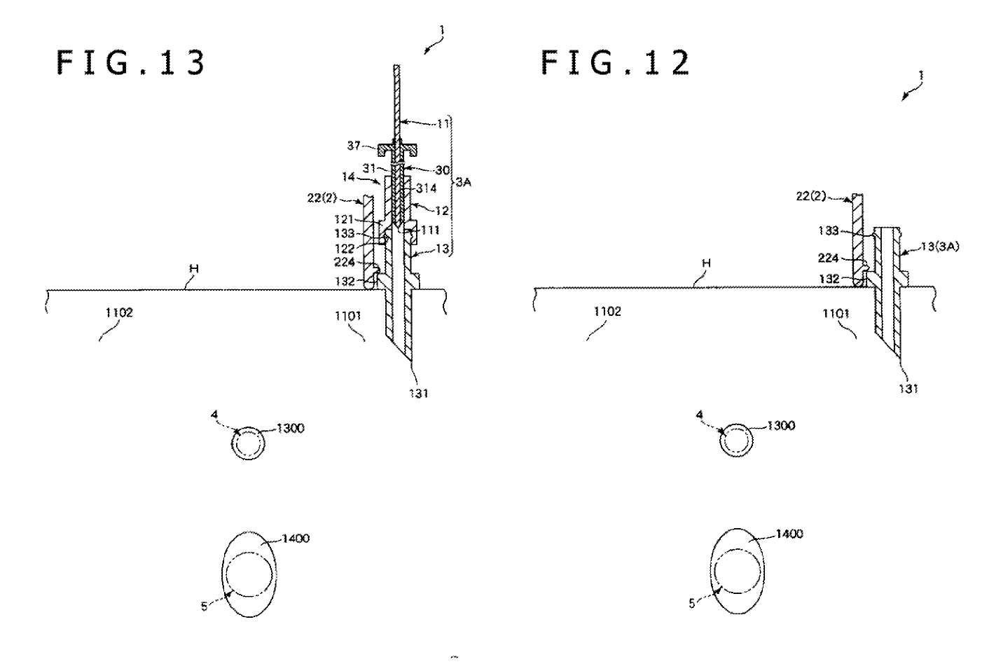

As shown in FIG. 12, the restriction member 13 is used in a state of being set indwelling at a body surface H (living body surface) by puncturing the body surface H, prior to puncturing by the puncture needle 11. Like the connection member 12, the restriction member 13 is composed essentially of a tubular body which is higher than the sheath 30 in rigidity. The tubular body has a rectilinear center axis. The restriction member 13 has a sharp puncture portion 131 at a distal end of the restriction member 13. The puncture portion 131 can make it possible to easily and assuredly puncture the body surface H toward one of left and right obturator foramens 1101 and 1102 (in this exemplary embodiment, the obturator foramen 1101). In accordance with an exemplary embodiment, the overall length of the restriction member 13 can be preferably equal to or greater than the overall length of the connection member 12.

In accordance with an exemplary embodiment, an inside diameter of the restriction member 13 can be slightly greater than the outside diameter of the main body 31 of the sheath 30, like the inside diameter of the connection member 12, which can help ensure that as shown in FIG. 13, the curved portion 314 is restricted into a rectilinear shape by the connection member 12 and, successively, restricted into a rectilinear shape (straightened) by the restriction member 13. In accordance with an exemplary embodiment, at its portion protruding from the distal opening of the restriction member 13, the curved portion 314 is released from the restriction by the restriction member 13, to return into a curved state (see FIGS. 14 to 16).

In accordance with an exemplary embodiment, the restriction member 13 can be provided with a flange portion 132 at an outer circumferential part of an intermediate portion in the longitudinal direction of restriction portion 132. The flange portion 132 is composed essentially of a diametrically enlarged portion where the outside diameter of the restriction member 13 is enlarged. As shown in FIG. 12, the flange portion 132 abuts on the body surface H when the body surface H is punctured by the restriction member 13, which helps restrict a depth of puncturing of the body surface H by the restriction member 13. In addition, in the puncture state wherein the body surface H is punctured by the restriction member 13, the flange portion 132 can engage with the engaging portion 224 of the guide portion 22 of the frame 2, and can be pressed against the body surface H, which can help prevent the restriction member 13 from being unwillingly pulled out of the body surface H. Accordingly, the puncture state, or indwelling state, can be reliably maintained.

In addition, at the outer circumferential portion of the proximal end of the restriction member 13, the projection 133 is projectingly formed along the circumferential direction. As disclosed above, the projection 133 can contribute to assured maintaining of the connected state of the connection member 12 and the restriction member 13, through the insertion of the projection 133 into the groove 122 formed in the diametrically enlarged portion 121 of the connection member 12.

The material constituting the restriction member 13 is not restricted; for example, the same material as that constituting the connection member 12 can be used.

The restriction member 13 has the puncture portion 131 in the present exemplary embodiment, this is not restrictive, and the restriction member 13 may be one that does not have the puncture portion 131.

In the puncture member 3A, a curvature-changing unit 14 is composed essentially of the sheath 30, the connection member 12 and the restriction member 13. The curvature-changing unit 14 is a mechanism changing a curvature of the puncture needle 11 in the process of puncturing a living body by the puncture needle 11. Then, a plurality of kinds of puncture can be obtained according to the curvature. In this exemplary embodiment, a first puncture with substantially zero curvature and a second puncture with a curvature greater than the curvature in the first puncture can be obtained.

In treatment of female urinary incontinence, the first puncture is performed during when the needle tip 111 of the puncture needle 11 is moved from the body surface H to a point of immediately before reaching the obturator foramen 1101 (see FIGS. 12 and 13). In this first puncture, the puncture needle 11 is inserted and passed in the sheath 30 to have its needle tip 111 protruding from the sheath 30, and, in this state, the puncture needle 11 passes through the connection member 12 and the restriction member 13. In this instance, the puncture needle 11 can be rectified into a rectilinear shape by the restriction member 13, together with the curved portion 314 of the sheath 30 such that the living body can be rectilinearly punctured, that is, to perform the first puncture.

Then, the second puncture is performed during when the needle tip 111 of the puncture needle 11 is moved past the obturator foramen 1101 and through a region between the urethra 1300 and the vagina 1400 and crosses (passes) the obturator foramen 1102 (the obturator foramen on the other side) (see FIG. 14). In this second puncture, the puncture needle 11 protrudes gradually from the restriction member 13, together with the curved portion 314 of the sheath 30. In this instance, the restriction by the restriction member 13 is lifted, and the curved portion 314 returns into the curved state. As a result, the puncture needle 11 follows the curved state of the curved portion 314, for example, lies along the curved shape of the curved portion 314, which punctures the living body in an arcuate shape, that is, to perform the second puncture.

In accordance with an exemplary embodiment, after the needle tip 111 of the puncture needle 11 crosses the obturator foramen 1102 and until the needle tip 111 protrudes from the body surface H, the first puncture is again performed (see FIG. 15). In this first puncture, a state is maintained in which the mounting portion 37 of the sheath 30 is mounted on the connection member 12 and movement of the sheath 30 is stopped, and only the puncture needle 11 is moved. In this case, the puncture needle 11 is also released from the restriction by the curved portion 314, and moves straight forward as it is, for example, and moves in a tangential direction in regard of the curved portion 314 such that the living body can be rectilinearly punctured, that is, to perform the first puncture.

In accordance with an exemplary embodiment, in this way, the curvature-changing unit 14 can enable the puncture needle 11 to be operated so as to sequentially perform the first puncture, the second puncture, and the first puncture in this order.

Meanwhile, depending on a body type of the patient, for example, in the case of a comparatively large patient, the puncture needle 11 may be curved in an arcuate shape immediately upon puncturing. In such a situation, the urethra 1300 is present on a forward side in an intended direction of travel of the needle tip 111. If the puncturing operation is continued under this condition, erroneous puncturing of the urethra 1300 can occur. In the puncture apparatus 1, however, the curvature-changing unit 14 can enable the first puncture to be performed until the needle tip 111 comes to a position avoiding the urethra 1300. Accordingly, the above disclosed erroneous puncturing can be relatively prevented.

In accordance with an exemplary embodiment, that while the curvature-changing unit 14 is composed essentially of the sheath 30, the connection member 12, and the restriction member 13 in this exemplary embodiment as disclosed above not restrictive. For example, the connection member 12 may be omitted.

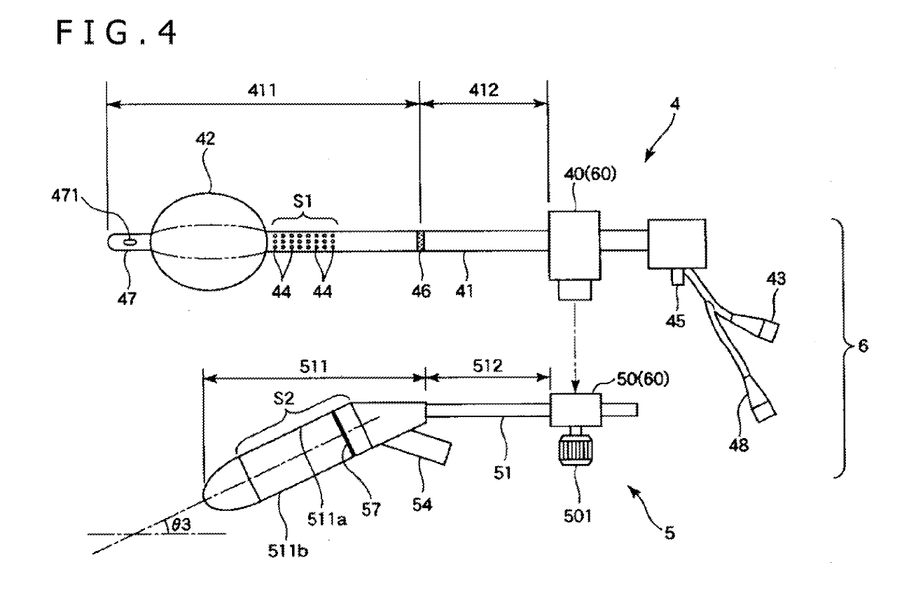

As shown in FIGS. 1 and 4, the insertion instrument 6 can include a urethral insertion portion (second insertion portion) 41 to be inserted into the urethra 1300, a vaginal insertion portion (first insertion portion) 51 to be inserted into the vagina 1400, and the support part 60 supporting the urethral insertion portion 41 and the vaginal insertion portion 51. As disclosed above, the insertion instrument 6 can be composed essentially of the urethral insertion member 4 and the vaginal insertion member 5. The urethral insertion member 4 has the urethral insertion portion 41, and the vaginal insertion member 5 has the vaginal insertion portion 51. In addition, the support part 60 can include a support portion 40 which is possessed by the urethral insertion member 4 and supports the urethral insertion portion 41 and a support portion 50 which is possessed by the vaginal insertion member 5 and supports the vaginal insertion portion 51. In the insertion instrument 6, the urethral insertion member 4 and the vaginal insertion member 51 can be freely detachable by way of the support portions 40 and 50, respectively.

In accordance with an exemplary embodiment, the urethral insertion member 4 can include the elongated urethral insertion portion 41 whose portion ranging from a distal end to an intermediate portion of the urethral insertion member 4 can be inserted into a urethra 1300, and the support portion 40 which supports the urethral insertion portion 41. In the following disclosure, for convenience of description, that portion of the urethral insertion member 4 which is located inside the urethra 1300 (inclusive of a bladder 1310) in the mounted state will be referred to also as “insertion portion 411,” whereas that portion of the urethral insertion member 4 which is exposed via a urethra orifice to the outside of the body in the mounted state and which ranges to the support portion 40 will be referred to also as “non-insertion portion 412.”

The urethral insertion portion 41 can be in the shape of a tube with its distal end rounded. In addition, the insertion portion 411 is provided at its distal portion with an inflatable and deflatable balloon 42 and a urine drain portion 47. The balloon 42 can function as a restriction portion restricting a position in an axial direction of the urethral insertion member 4 in the inside of the urethra 1300. For example, when the puncture apparatus 1 is used, the balloon 42 can be inflated after inserted into a patient's bladder 1310. Then, with the balloon 42 caught on a bladder neck, the position of the urethral insertion member 4 relative to the bladder 1310 and the urethra 1300 can be fixed. For example, in accordance with an exemplary embodiment, the urine drain portion 47 can be used for draining urine present inside the bladder 1310.

The balloon 42 can extend through the inside of the urethral insertion portion 41, to be connected to a balloon port 43 provided at a proximal portion of the urethral insertion portion 41. A balloon-inflating instrument such as a syringe can be connected to the balloon port 43. When a working fluid (a liquid such as physiological salt solution, or a gas or the like) is supplied from the balloon-inflating instrument into the balloon 42, the balloon 42 is inflated. On the contrary, when the working fluid is drawn out of the balloon 42 by the balloon-inflating instrument, the balloon 42 is deflated. In FIG. 4, the balloon 42 in its deflated state is drawn in two-dot chain line, whereas the balloon 42 in its inflated state is drawn in solid line.

In accordance with an exemplary embodiment, the urine drain portion 47 can be provided with a drain hole 471 providing communication between an inside and an outside of the urine drain portion 47. In addition, the urine drain portion 47 can extend through the inside of the urethral insertion portion 41, to be connected to a urine drain port 48 provided at a proximal portion of the urethral insertion portion 41. Thus, in accordance with an exemplary embodiment, the urine introduced through the drain hole 471 into the urine drain portion 47 can be drained via the urine drain port 48.

The balloon 42 and the urine drain portion 47 can be configured by use of a double lumen, for example.

In addition, the insertion portion 411 is formed with a plurality of suction holes 44 at an intermediate portion of the insertion portion 411. The plurality of suction holes 44 can be laid out over a whole range in a circumferential direction of the urethral insertion portion 41. Each of the suction holes 44 is connected to a suction port 45 provided at a proximal portion of the urethral insertion portion 41, via the inside of the urethral insertion portion 41. A suction device such as a pump can be connected to the suction port 45. When the suction device is operated in a state wherein the urethral insertion portion 41 is inserted in the urethra 1300, a urethral wall can be sucked and fixed onto the urethral insertion portion 41. When the urethral insertion portion 41 is pushed in toward the distal end (toward the inside of the body) under this condition, the urethra 1300 is also pushed in together with the urethral insertion portion 41. As a result, for example, the bladder 1310 can be shifted to such a position as not to overlap with a puncture route for the puncture member 3A, whereby the puncture route for the puncture member 3A can be secured. Therefore, puncturing by the puncture member 3A can be carried out relatively accurately and relatively safely. The number of the suction holes 44 is not limited; for example, the number may be one. Besides, a layout of the suction holes 44 is not restricted, for example, the suction holes 44 may be formed in only a part of the range in the circumferential direction of the urethral insertion portion 41.

In addition, at a boundary between the insertion portion 411 and the non-insertion portion 412, a marker 46 can be provided with which to check a depth of insertion of the urethral insertion portion 41 into the urethra 1300. When the urethral insertion portion 41 is inserted in the urethra 1300 and the balloon 42 is located inside the bladder 1310, the marker 46 is located at the urethral orifice, which permits relatively easy checking of the depth of insertion of the insertion portion 411 into the urethra 1300. The marker 46 can be necessary only to be externally visible, and can be composed essentially of, for example, a colored portion, a recessed and projected portion, or the like. In accordance with an exemplary embodiment, a graduation with indications of distance from the distal end of the urethral insertion portion 41 may be provided, in place of the marker 46.

A length of the insertion portion 411 is not limited, and may be set, as necessary, for example, according to a length of the urethra 1300 and a shape of the bladder 1310 of the patient, or the like. In accordance with an exemplary embodiment, the length of the insertion portion 411, for example, is preferably about 50 to 100 mm, in view of the fact that the length of a female urethra 1300 is generally about 30 to 50 mm.

A length of the non-insertion portion 412 (a spacing between the urethral orifice and the support portion 40) is not limited. For example, the length is preferably not more than about 100 mm, preferably in the range of about 20 to 50 mm. By such a setting, the length of the non-insertion portion 412 can be made appropriate, which can enhanced operability. For example, if the length of the non-insertion portion 412 exceeds the above-mentioned upper limit, a center of gravity of the puncture apparatus 1 would, depending on the configuration of the frame 2 or the like factors, can be largely deviated from the patient, possibly leading to a lowered stability of the puncture apparatus 1 in the mounted state.

The material constituting the urethral insertion member 4 is not restricted. Examples of the material applicable here include various metallic materials such as stainless steels, aluminum, aluminum alloys, titanium, titanium alloys, etc. and various resin materials.

In accordance with an exemplary embodiment, an inclination angle θ2 of the plane f9 relative to a plane f2 orthogonal to an axis J2 of the urethral insertion portion 41 can be, for example, about 20 to 60 degrees, more preferably about 30 to 45 degrees, and further preferably about 35 to 40 degrees. For example, the main body 31 is preferably set indwelling in the body so that the angle formed between the plane f9 and a plane orthogonal to the axis of the urethra 1300 is, for example, about 20 to 60 degrees, more preferably about 35 to 45 degrees, and further preferably about 30 to 45 degrees. In accordance with an exemplary embodiment, such a setting makes it possible to easily perform the puncturing by the puncture member 3A and to make shorter the distance of puncture by the puncture member 3A.

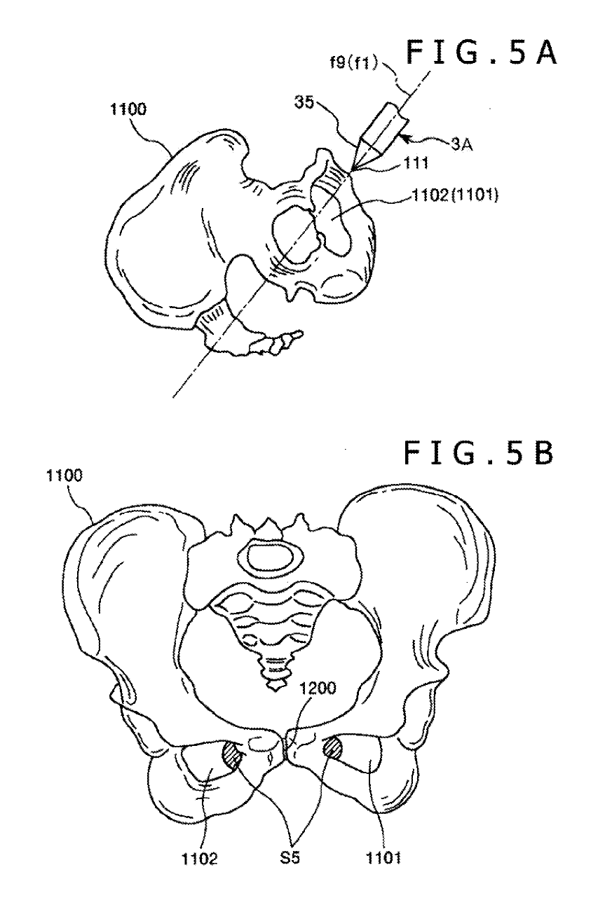

In accordance with an exemplary embodiment, setting the inclination angle θ2 to within the above-mentioned range can help ensure that the puncture member 3A can capture the left and right obturator foramens 1101 and 1102 of the pelvis 1100 wider on a planar basis, so that a wide puncturing space for the puncture member 3A can be secured. For example, in a condition where a patient is put in a predetermined posture (lithotomy position), puncturing by the puncture member 3A can be performed in a direction comparatively nearer to a perpendicular direction relative to the obturator foramens 1101 and 1102. Therefore, the puncturing by the puncture member 3A can be carried out relatively easily. In addition, since the puncturing by the puncture member 3A is performed in a direction comparatively nearer to the perpendicular direction relative to the obturator foramens 1101 and 1102, it can help ensure that the puncture member 3A passes through a shallow part of tissue, so that the puncture member 3A can cross a region between the left and right obturator foramens 1101 and 1102 by passing a short distance. Accordingly, as shown in FIG. 5B, the puncture member 3A can be passed through those regions of the obturator foramens 1101 and 1102 which are near a pubic symphysis 1200, for example, through safety zones S5. Since safety zones S5 are regions where there are few nerves and blood vessels the damage to which is to be obviated, puncturing by the puncture member 3A can be performed relatively safely. In addition, the result is relatively minimal invasiveness, and whereby burden on the patient can be relatively suppressed. Thus, with the inclination angle θ2 set to within the above-mentioned range, puncturing of a patient by the puncture member 3A can be appropriately performed. In addition, by puncturing at the above-mentioned angle, it is facilitated to aim at a tissue between a middle-part urethra (which refers to an intermediate part in a lengthwise direction of the urethra 1300) and the vagina 1400. The position between the middle-part urethra and the vagina 1400 is a position suitable as a site where to embed the implant 9 for treatment of urinary incontinence.

In accordance with an exemplary embodiment, for example, in the case where the inclination angle θ2 is less than the aforementioned lower limit or in excess of the aforementioned upper limit, there may arise an issue in that, depending on an individual differences concerning the patient or a posture during the procedure or the like factors, the puncture member 3A cannot capture the obturator foramens 1101 and 1102 wide on a planar basis or the puncture route cannot be made sufficiently short.

In accordance with an exemplary embodiment, for example, the puncturing can be conducted in a condition where either one or both of the urethra 1300 and the vagina 1400 are positionally shifted so as to be pushed toward the inside of the body. Such an operation help permits relatively easy puncturing of the tissue between the middle-part urethra and the vagina 1400. The method pushing in either one of the urethra 1300 and the vagina 1400 toward the inside of the body may be, for example, a method in which the urethral insertion member 4 and/or the vaginal insertion member 5 is inserted into an appropriate position, then, in this condition, the urethra 1300 and/or the vagina 1400 is sucked by the suction holes 44 and 59 (disclosed later) possessed by these members 4 and 5, and thereafter the urethral insertion member 4 and/or the vaginal insertion member 5 is further moved toward the inside of the body along the axis thereof until reaching a predetermined position. In the condition where at least one of the urethra 1300 and the vagina 1400 has thus been positionally shifted so as to be pushed in toward the inside of the body, the main body 31 of the sheath 30 is made to puncture the living body perpendicularly to the left and right obturator foramens 1101 and 1102 of the pelvis, whereby a passage can be formed in a position suited to implanting of the implant 9.

In accordance with an exemplary embodiment, a setting can be made to cause an orbital path of the main body 31 of the sheath 30 to pass the safety zones S5 of the left and right obturator foramens 1101 and 1102 of the pelvis, at least one of the urethra 1300 and the vagina 1400 is positionally shifted toward the inside of the body so as to locate the orbital path between the middle-part urethra and the vagina 1400, and puncturing is performed along the orbital path of the main body 31, thereby forming the passage.

As shown in FIGS. 1 and 4, the vaginal insertion member 5 can include the elongated vaginal insertion portion (first insertion portion) 51 whose portion from a distal end to an intermediate portion of vaginal insertion portion 51 is inserted into a vagina 1400 and the support portion 50 supporting the vaginal insertion portion 51. In the following, for convenience of description, that portion of the vaginal insertion member 5 which is located in the vagina 1400 in the mounted state will be referred to also as “insertion portion 511,” and that portion of the vaginal insertion member 5 which is exposed via a vaginal orifice to the outside of the living body in the mounted state and which ranges to the support portion 50 will be referred to also as “non-insertion portion 512.”

In accordance with an exemplary embodiment, the insertion portion 511 can be elongated. In addition, the insertion portion 511 can extend at an inclination relative to the insertion portion 411 so that the insertion portion 511 is spaced from the insertion portion 411 on the distal end. With the insertion portion 511 inclined relative to the insertion portion 411, a positional relationship between the insertion portions 411 and 511 can be set closer to the positional relationship between the urethra 1300 and the vagina 1400, as compared with the case where the insertion portion 511 is not inclined in this way. In the mounted state, the puncture apparatus 1 can be stably held onto the patient, and the relative burden on the patient can be mitigated. An inclination angle θ3 of the insertion portion 511 relative to the insertion portion 411 is not limited; for example, the inclination angle θ3 can be about 0 to 45 degrees, more preferably about 0 to 30 degrees, which can enable the above-mentioned effect to be displayed conspicuously. In accordance with an exemplary embodiment, in the case where the inclination angle θ3 is less than the aforementioned lower limit or in excess of the aforementioned upper limit, there may arise an issue in that, depending on individual differences concerning the patient or the posture during the procedure or the like factors, the urethra 1300 and/or the vagina 1400 may be deformed unnaturally in the mounted state, possibly hampering the puncture apparatus 1 from being stably held.

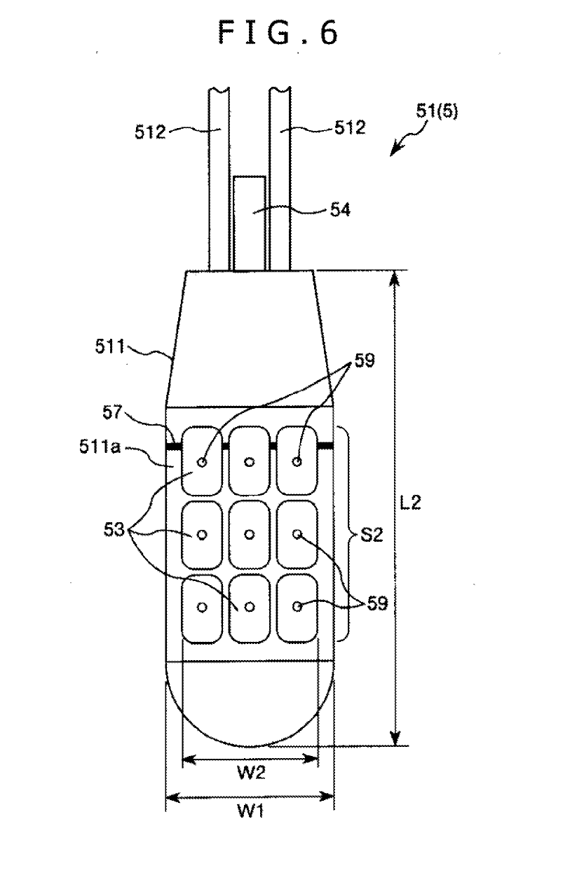

As shown in FIG. 6, the insertion portion 511 is in a flat shape collapsed in a vertical direction of the puncture apparatus 1 (in an array direction of the urethra 1300 and the vagina 1400). In addition, the insertion portion 511 has a central portion having a substantially constant width and a somewhat rounded distal portion. A length L2 of the insertion portion 511 is not limited, and can be, for example, about 20 to 100 mm, more preferably about 30 to 60 mm. A width W1 of the insertion portion 511 is not restricted, and can be, for example, about 10 to 40 mm, more preferably about 20 to 30 mm. In addition, a thickness of the insertion portion 511 is not limited, and can be, for example, about 5 to 25 mm, more preferably about 10 to 20 mm. Set to have such length, width, and thickness, as disclosed, the insertion portion 511 can be suited in shape and size to most vaginas. Therefore, stability of the puncture apparatus 1 in the mounted state can be enhanced, and burden on the patient can be relatively alleviated.

In addition, an upper surface (a surface on the urethral insertion portion 41 side) 511a of the insertion portion 511 can be formed with a plurality of bottomed recesses 53. The number of the recesses 53 is not limited; for example, the number may be one. In accordance with an exemplary embodiment, each recess 53 can be provided with a single suction hole 59 in its bottom surface. Each suction hole 59 can be connected to a suction port 54 provided at a proximal portion of the insertion portion 511, through the inside of the insertion portion 511. The suction port 54 can also be located in the outside of the living body in the mounted state. A suction device such as a pump can be connected to the suction port 54. When the suction device is operated in the condition where the insertion portion 511 is inserted in the vagina 1400, an anterior wall of vagina, 1410, which is an upper surface of a vaginal wall, is sucked and fixed onto the insertion portion 511. When the vaginal insertion portion 51 with the vaginal wall is sucked and fixed thereon is pushed toward the distal end (toward the inside of the body), the vaginal wall can be pushed in together with the vaginal insertion portion 51. Thus, a configuration and a shape of the vaginal wall, which can help secure a puncture route for the puncture member 3A can be obtained, and the puncture member 3A can accurately and safely perform the puncturing as disclosed herein.

A region S2 in which the plurality of recesses 53 are formed is disposed opposite to a region S1. The needle tip 111 of the puncture member 3A passes between these regions S1 and S2. Since the urethra 1300 (which is a lower surface of the urethral wall) is sucked onto the insertion portion 411 in the region S1 and the anterior wall of vagina, 1410, is sucked onto the insertion portion 511 in the region S2, as above-mentioned, the urethral wall and the vaginal wall are spaced wider apart from each other between the regions S1 and S2. By causing the puncture member 3A to pass such a region, therefore, the puncturing by the puncture member 3A can be performed safely.

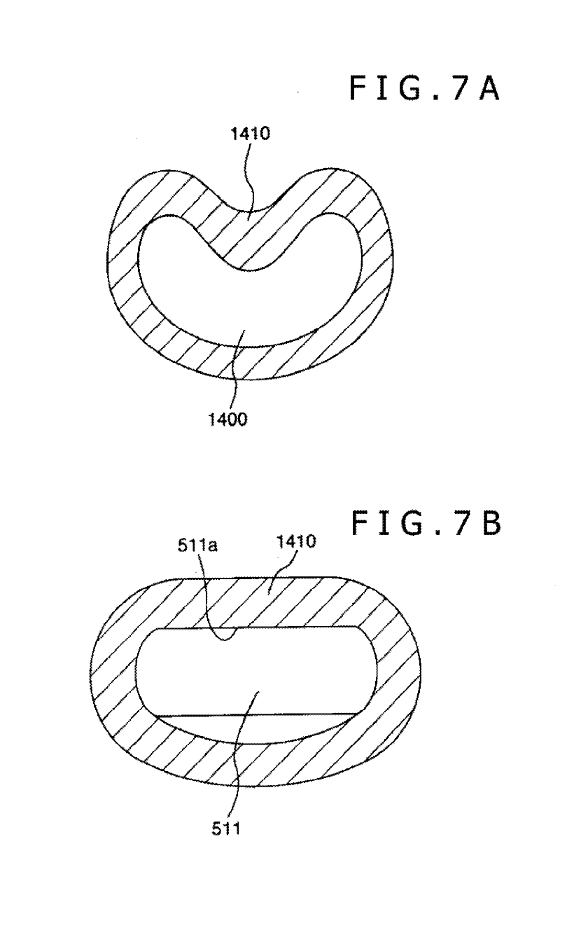

The region S2 ranges over substantially a whole region in a width direction of the upper surface 511a. A width W2 of the region S2 is not limited, and can be, for example, about 9 to 39 mm, more preferably about 19 to 29 mm, which can help enable the anterior wall of vagina, 1410, to be reliably sucked onto the insertion portion 511, without being much influenced by the shape of the vaginal wall. For example, in some patients, the vagina 1400 may be so shaped that part of the anterior wall of vagina, 1410, is hanging down into the inside of the vagina 1400, as shown in FIG. 7A. Even in such a case, setting the width W2 to within the above-mentioned range ensures that not only the hanging-down portion but also the portions on both sides of the hanging-down portion can be sucked in an assured manner, as shown in FIG. 7B. Therefore, the anterior wall of vagina, 1410, can be reliably spaced from the urethra 1300, without being affected by the shape of the vagina 1400. For example, in this exemplary embodiment, the insertion portion 511 is flat-shaped, so that the anterior wall of vagina, 1410, can be sucked so as to be spaced apart from the urethra 1300. In accordance with an exemplary embodiment, the biological tissue between the urethral wall and the vaginal wall can be expanded (spread).

In addition, the insertion portion 511 can be include a marker (puncture position checking portion) 57 with which a puncture route for the puncture apparatus 1 can be checked. For example, the puncture apparatus 1 can be so fixed as to puncture a region (biological tissue) between the vaginal wall present on an upper side of the position where the marker 57 is located and the urethral wall. Therefore, operability and safety of the insertion instrument 6 can be enhanced. The marker 57 can be provided at least on a lower surface 511b of the insertion portion 511. The lower surface 511b is a surface which is oriented toward the vaginal orifice can be visually confirmed by the operator via the vaginal orifice, in the mounted state. With the marker 57 provided on the lower surface 511b, the puncture route for the puncture apparatus 1 can be reliably checked. In addition, the depth of insertion of the insertion portion 511 into the vagina can be checked. The marker 57 is necessary only to be externally visible, and can be configured by use of, for example, a colored portion, a recessed and projected portion, or the like.

In accordance with an exemplary embodiment, the non-insertion portion 512 can be in the shape of a thin bar which extends substantially parallel to the urethral insertion portion 41. A spacing D between the non-insertion portion 512 and the urethral insertion portion 41 is not limited, and can be, for example, about 5 to 40 mm, correspondingly to the spacing between the urethral orifice and the vaginal orifice in common women.

A length of the non-insertion portion 512 (a spacing between the vaginal orifice and the support portion 50) is not restricted, and can be, for example, not more than about 100 mm, preferably in the range of about 20 to 50 mm, which can permit the non-insertion portion 512 to be appropriate in length, leading to enhanced operability. For example, if the length of the non-insertion portion 512 exceeds the above-mentioned upper limit, the center of gravity of the puncture apparatus 1 would, depending on the configuration of the frame 2 or the like factors, be largely deviated from the patient, possibly leading to a lowered stability of the puncture apparatus 1 in the mounted state.

The support portion 50 can be provided with a male screw 501. With the male screw 501 fastened into a female screw (not shown) formed in the support portion 40, the support portions 40 and 50 can be fixed to each other.

The material constituting the vaginal insertion member 5 is not restricted. Examples of the material applicable here include various metallic materials such as stainless steels, aluminum, aluminum alloys, titanium, titanium alloys, etc. and various resin materials, like the examples of the material for the urethral insertion member 4.

In accordance with an exemplary embodiment, the urethral insertion member 4 and the vaginal insertion member 5 constituting the insertion instrument 6 can be freely detachable in the puncture apparatus 1, this configuration is not restrictive. For example, a configuration may be adopted in which the urethral insertion member 4 and the vaginal insertion member 5 are non-detachable.

In addition, while the urethral insertion portion 41 is fixed to the support portion 40 in the puncture apparatus 1, this configuration is not restrictive. For example, a configuration may be adopted wherein the urethral insertion portion 41 can be selectively switched between a state of being fixed to the support portion 40 and a state of being slidable in the axial direction relative to the support portion 40. In accordance with an exemplary embodiment, for example, a configuration may be adopted wherein untightening of a screw provided on the support portion 40 can permit the urethral insertion portion 41 to be slid relative to the support portion 40, whereas tightening the screw can render the urethral insertion portion 41 fixed to the support portion 40. This configuration can enable regulation of the length of the non-insertion portion 412, which makes the insertion instrument 6 user-friendly. In accordance with an exemplary embodiment, this can also apply to the vaginal insertion portion 51.

In accordance with an exemplary embodiment, the members can be fixed to the frame 2 so that the inclination angle θ2 will be constant in the puncture apparatus 1, and wherein this configuration is not restrictive. Thus, the inclination angle θ2 may be variable, which can permit the inclination angle θ2 to be controlled according to the patient to be treated, which can improve the utility of the puncture apparatus 1.

In accordance with an exemplary embodiment, the implant 9 can be used together with the puncture apparatus 1 as disclosed below. As shown in FIG. 15, the implant (biological tissue-supporting indwelling body) 9 is an embeddable (implantable) instrument for treatment of female urinary incontinence, for example, an instrument supporting the urethra 1300. For example, when the urethra 1300 tends to move toward the vaginal wall side, the implant 9 supports the urethra 1300 in such a manner as to restrict its movement in a direction of coming away from the vaginal wall. As the implant 9, for example, a flexible elongate body can be used.

The implant 9 can include an implant main body (band-shaped elongate body) 91, and a bag-shaped wrapping material 92 accommodating the implant main body 91. In addition, the implant main body 91 can include a main body portion 911, and a band 912 interlocked to one end of the main body portion 911. With the implant 9 provided with the wrapping material 92, contamination of the implant main body 91 can be effectively prevented. In accordance with an exemplary embodiment, a guide wire, braid, string or the like may be used in place of the band 912.

In accordance with an exemplary embodiment, the main body portion 911 can be a reticulated band body. The main body portion 911 may be composed, for example, of a body obtained by intersecting linear members and knitting them into a reticulated form, for example, a reticulated braiding. Examples of the linear member include those which are circular in cross-sectional shape, those which are flat-shaped in cross section, etc.

The materials constituting the main body portion 911, the band 912 and the wrapping material 92 are each not restricted. Examples of the materials applicable here can include various biocompatible resin materials such as polypropylene, polyesters, or nylon, and fibers, etc.

The implant 9 is not restricted to the above-mentioned reticulated body, insofar as it can produce an effect equivalent to the aforementioned.

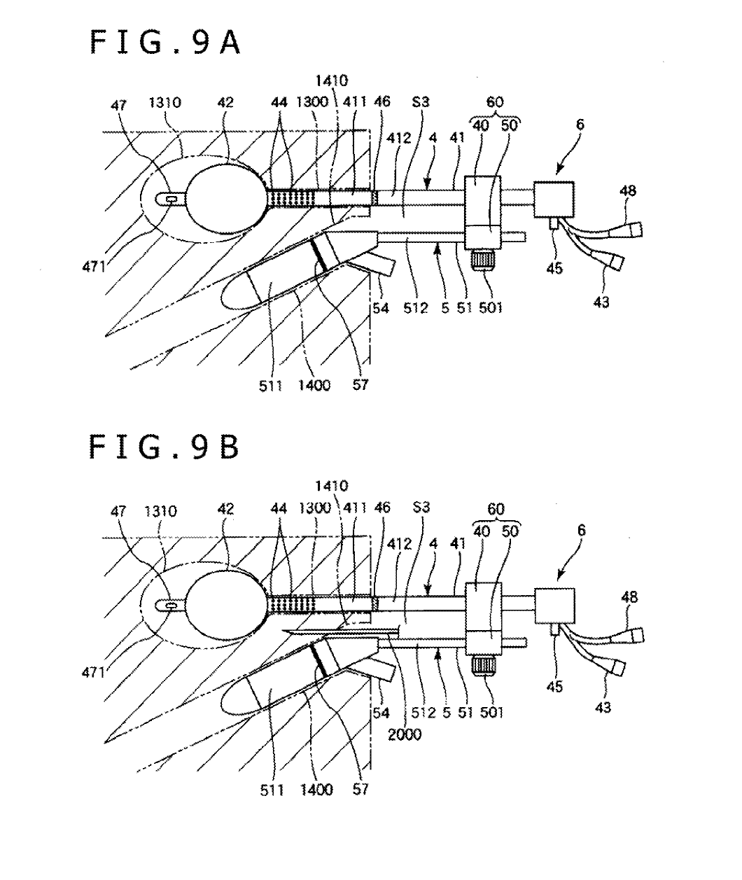

A method of using the puncture apparatus 1 (operation procedure) is disclosed, and described below referring to FIGS. 9A to 20. By use of the puncture apparatus 1 (by the puncturing method) a bow-like puncture hole can be formed by placing the implant 9 indwelling in a biological tissue, prior to placement of the implant 9 between the urethra 1300 and the vagina 1400.

First, a patient is put in a lithotomy position on an operating table, and the insertion instrument 6 is mounted onto the patient, as shown in FIG. 9A. Specifically, first, the urethral insertion portion 41 of the urethral insertion member 4 is inserted into the patient's urethra 1300. In this instance, while checking the depth of insertion by observing the marker 46, the balloon 42 is disposed inside the bladder 1310. The urethra 1300 is rectified into a predetermined shape by the urethral insertion portion 41 having the predetermined shape. In the case of this exemplary embodiment, the urethra 1300 can be rectified into a rectilinear shape by the urethral insertion portion 41 having the rectilinear shape.

Next, the balloon 42 is inflated, and, if necessary, urine is drained from the inside of the bladder 1310 through the drain hole 471. In addition, the vaginal insertion portion 51 of the vaginal insertion member 5 can be inserted into the patient's vagina 1400. In this instance, while checking the puncture position by observing the marker 57, the vaginal insertion portion 51 can be inserted to an appropriate depth. Then, the male screw 501 is operated, to fix the support portions 40 and 50. By this, mounting of the insertion instrument 6 onto the patient is completed. In this state, the non-insertion portions 412 and 512 are spaced from each other, the support part 60 is spaced from the body surface between the urethral orifice and the vaginal orifice, and that body surface is exposed. In addition, in the case where the insertion portion 511 and the anterior wall of vagina, 1410, are spaced from each other and a gap (space) is formed therebetween, a space S3 causing a syringe to puncture from the body surface between the urethral orifice and the vaginal orifice to a biological tissue between the urethra and the vagina is formed.

Subsequently, suction devices can be connected to the suction ports 45 and 54, and the suction devices are operated, whereby the urethra 1300 is sucked onto the urethral insertion portion 41, and the anterior wall of vagina, 1410, is sucked onto the vaginal insertion portion 51. For example, when the urethra 1300 is properly sucked onto the urethral insertion portion 41, the suction holes 44 are closed with the urethral wall, so that the suction via the suction port 45 is stopped or weakened. Similarly, when the anterior wall of vagina, 1410, is properly sucked onto the vaginal insertion portion 51, the suction holes 59 are closed with the vaginal wall, so that the suction via the suction port 54 is stopped or weakened. Therefore, based on the conditions of suction via the suction ports 45 and 54 (for example, the magnitude of sounds generated attendant on the suction via the suction ports 45 and 54), the operator can check whether or not the urethra 1300 and the anterior wall of vagina, 1410, have been properly sucked onto the urethral insertion portion 41 and the vaginal insertion portion 51, respectively. In accordance with an exemplary embodiment, the insertion instrument 6 may be provided with a checking mechanism for mechanical checking of the sucked state. The checking mechanism is not restricted, insofar as it enables checking of the sucked state. For example, the checking mechanism may be configured to include a flow measurement unit (negative-pressure sensor) measuring a flow rate through the suction port 54 and a decision unit deciding whether or not the sucking is being properly done, based on the measurement results sent from the flow measurement unit.

Next, humoral dissection can be performed. In accordance with an exemplary embodiment, as shown in FIG. 9B, the puncture needle of a syringe 2000 is caused to puncture the anterior wall of vagina, 1410, by way of the space (space S3) between the insertion portion 511 and the anterior wall of vagina, 1410, and a liquid such as physiological salt solution or local anesthetic is injected into the biological tissue between the urethra 1300 and the vagina 1400 (between the regions S1 and S2). As a result, the biological tissue between the regions S1 and S2 is expanded, the urethra 1300 is pressed against the urethral insertion portion 41, and the anterior wall of vagina, 1410, is pressed against the vaginal insertion portion 51.

In accordance with an exemplary embodiment, the suction through the suction holes 44 and 59 can be continuously conducted also during the humoral dissection. When the urethra 1300 is pressed against the urethral insertion portion 41 due to the humoral dissection, the urethra 1300 is further sucked onto the urethral insertion portion 41, so that the suction via the suction port 45 is stopped or weakened. Similarly, when the anterior wall of vagina, 1410, is pressed against the vaginal insertion portion 51, the anterior wall of vagina, 1410, is further sucked onto the vaginal insertion portion 51, so that the suction via the suction port 54 is stopped or weakened. Based on the conditions of suction via the suction ports 45 and 54, therefore, the operator can check whether the humoral dissection has been properly performed.

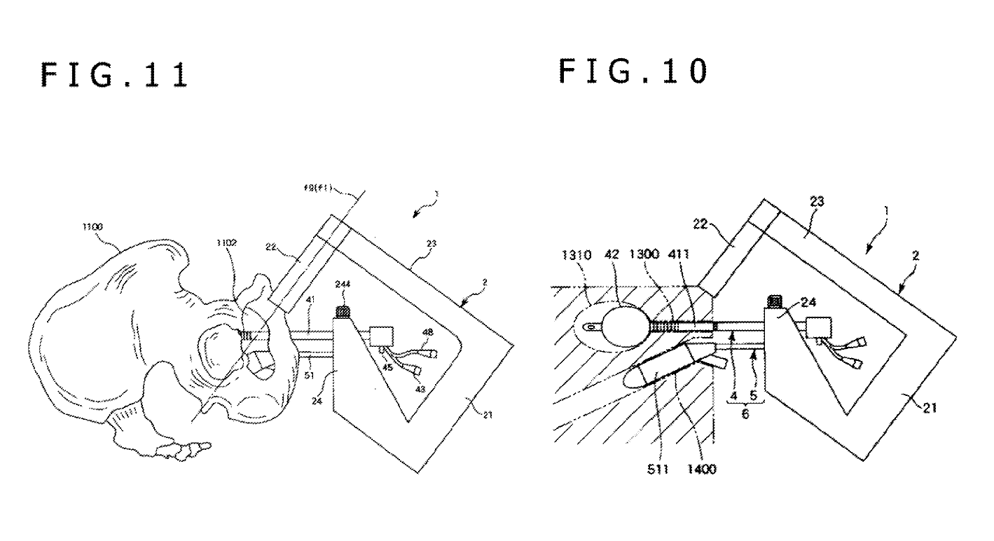

After the humoral dissection is conducted so that the urethra 1300 and the anterior wall of vagina, 1410, are thereby sufficiently spaced from each other, the restriction member 13 is set indwelling on the body surface H at a right-hand inguinal region of the patient or at a region near the inguinal region. Then, as shown in FIG. 10, the insertion instrument 6 can be fixed to the frame 2, which can result in the puncture apparatus 1 being mounted on the patient. In this state, a positional relationship between the pelvis 1100 and the puncture apparatus 1 is as shown in FIG. 11. In addition, as shown in FIG. 12, a flange portion 132 of the restriction member 13 can be engaged with the engaging portion 224 of the guide portion 22 of the frame 2, which can result in the restriction member 13 being pressed against and fixed to the body surface H. Furthermore, the connection member 12 can be connected to the restriction member 13.

Next, for example, while grasping the grasping portion 21 or the interlock portion 23 of the frame 2 by one hand, the puncture needle 11 inserted and passed in the sheath 30 is pushed into the restriction member 13 through the connection member 12, as shown in FIG. 13, by the other hand. In this instance, the needle tip 111 of the puncture needle 11 is slightly protruding from the distal opening portion of the sheath 30.

Then, as the puncture needle 11 is gradually pushed into the sheath 30, the first puncture is performed as disclosed above.

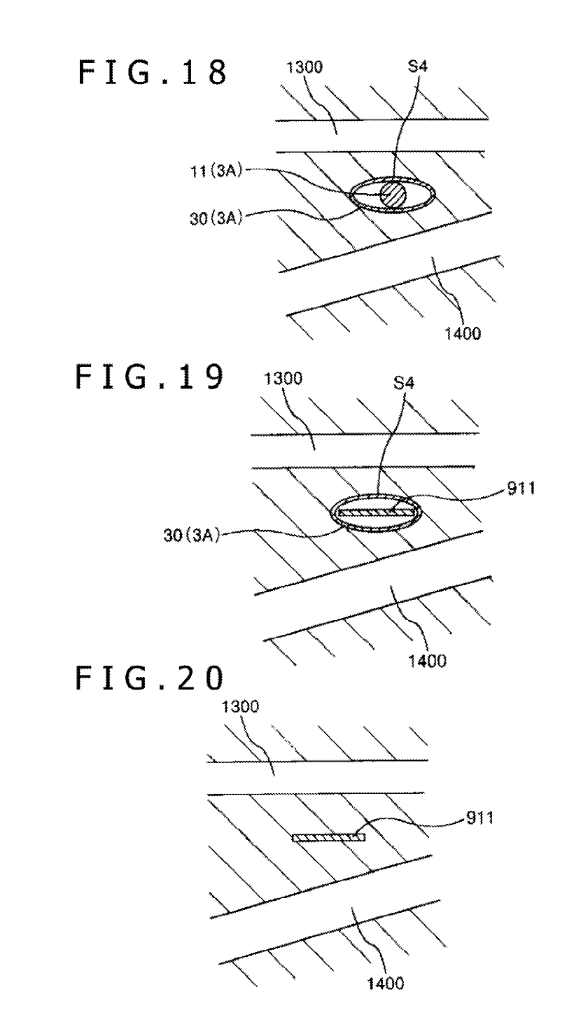

When the operation of pushing the puncture needle 11 together with the sheath 30 is continued further, the second puncture is performed as above-mentioned. As shown in FIG. 14, the puncture route of the puncture needle 11 in the second puncture is a route of passing the obturator foramen 1101, the region between the urethra 1300 and the vagina 1400, and the obturator foramen 1102. In this instance, between the urethra 1300 and the vagina 1400, the puncture needle 11 and the sheath 30 are in a state as shown in FIG. 18. In accordance with an exemplary embodiment, the second puncture can be performed until the mounting portion 37 of the sheath 30 is mounted on the connection member 12 and movement of the sheath 30 is thereby stopped.

Subsequently, as shown in FIG. 15, only the puncture needle 11 is gradually pushed in, whereby the first puncture is again performed as disclosed above. Because of this puncture, the needle tip 111 protrudes from the body surface H at the left-hand inguinal region or at a region near the inguinal region to the outside of the body. Thereafter, while taking the implant main body 91 out of the wrapping material 92, the band 912 is passed through a through-hole (not shown) provided in the vicinity of the needle tip 111 of the puncture needle 11, which can result in the implant main body 91 and the puncture needle 11 being interlocked with each other.

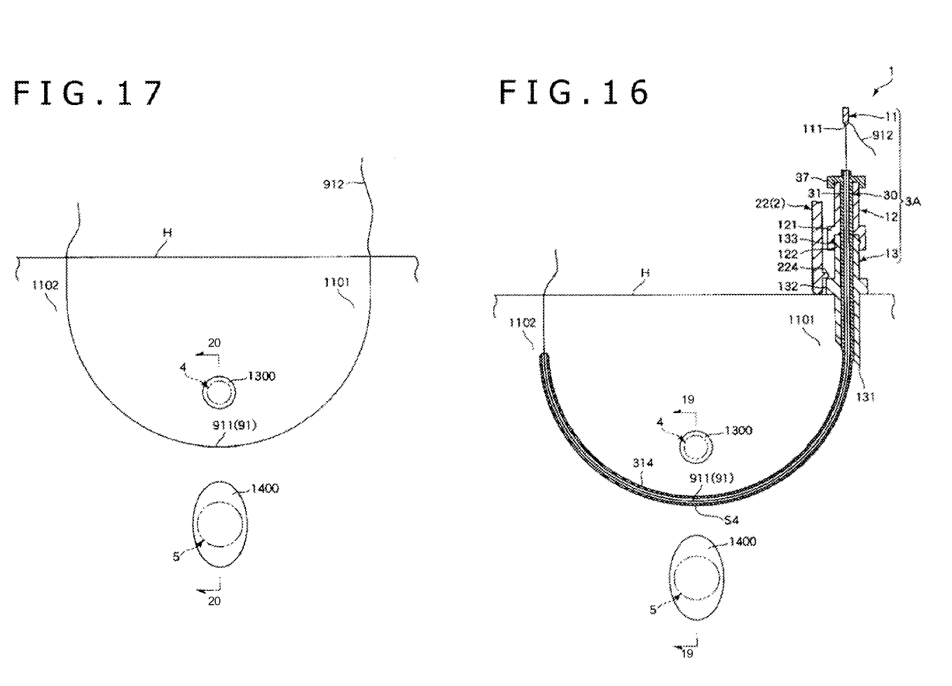

Next, as shown in FIG. 16, while keeping the sheath 30 indwelling in the living body, the puncture needle 11 can be pulled in the reverse direction to the aforementioned, to be pulled out of the sheath 30, which can cause the implant main body 91 to be pulled together with the puncture needle 11 and be inserted and passed in the sheath 30, in place of the puncture needle 11. Thus, the implant main body 91 can be kept accommodated in the wrapping material 92 until immediately before being disposed within the sheath 30, such that contamination of the implant main body 91 can be relatively prevented.

In addition, if necessary, the position of the sheath 30 can be put into working order. For example, to shift the sheath 30 toward the proximal end or the distal end so that the central portion S4 of the main body 31 will be located between the urethra 1300 and the vagina 1400 as assuredly as possible. This results in that, as shown in FIG. 19, the central portion S4 is disposed with its width direction (the direction of the major axis J32) substantially in parallel to the urethra 1300. Specifically, the urethra 1300 straightened by the insertion of the urethral insertion member 4 therein and the width direction of the central portion S4 are located substantially in parallel to each other. Furthermore, since the central portion S4 is flat-shaped, it is preferable that a posture of the main body portion 911 of the implant main body 91 follows the flat shape. In other words, as shown in FIG. 19, the main body portion 911 is disposed within the sheath 30 in such a manner that its width direction coincides with the width direction of the central portion S4. As for relationship with the urethra 1300, the implant main body 91 is disposed in parallel to the straightened urethra 1300.

Subsequently, as shown in FIG. 17, the puncture needle 11 is taken out of the implant main body 91, and the frame 2 is detached from the patient. Besides, the sucking of the urethra 1300 by the urethral insertion portion 41 and the sucking of the anterior wall of vagina, 1410, by the vaginal insertion portion 51 are stopped. Because of this, the positions and shapes of the urethra 1300 and the vagina 1400 are returned into their original natural state.

Thereafter, the sheath 30, the restriction member 13, and the connection member 12 are collectively drawn out of the living body. As the sheath 30 is gradually taken out of the living body, the surrounding tissue having been forced spread or expanded by the sheath 30 returns into an original position, and the tissue comes into contact with the implant main body 91 gradually from one end side toward the other end side of the implant main body 91, which can help ensure that the implant main body 91 can be placed indwelling in an as-is state, with no unnecessary tensile force exerted on implant main body 91, and thus no need for control of tension on the implant main body 91. Because of these operations, the implant main body 91 is embedded in the living body, as shown in FIG. 20. In the state wherein the implant main body 91 is embedded in the living body, the main body portion 911 is disposed substantially parallel to the urethra 1300, in the region between the urethra 1300 and the vagina 1400. Consequently, the urethra 1300 can be supported by the implant main body 91 in a wider area.

Next, the urethral insertion member 4 is pulled out of the urethra 1300, and the vaginal insertion member 5 is pulled out of the vagina 1400. After the urethral insertion member 4 is pulled out, the urethra 1300 returns into its shape in the natural state. In this case, for example, since the main body portion 911 is left embedded in the tissue, the urethra 1300 in the natural state and the main body portion 911 can be maintained in a mutually parallel state. Thereafter, unnecessary portions of the implant main body 91 can be cut away, to complete the procedure. Since the sheath 30 is pulled out of the living body in the state wherein the urethral insertion member 4 is inserted in the urethra 1300, it can help prevent an excessive tension from being exerted on the urethra 1300 by the implant main body 91 placed indwelling in the living body.

As above-mentioned, according to the puncture apparatus 1, it is possible, in the process of puncturing a biological tissue to form a puncture hole, to appropriately change the curvature of the puncture needle 11 according to the depth of puncture. With the curvature of the puncture needle 11 thus varied, the puncture hole formed also can have a curvature varied along the longitudinal direction of puncture apparatus 1, which can help ensure that the puncture hole is in the form of reliably passing between the urethra 1300 and the vagina 1400. In this way, the puncture apparatus 1 helps ensure that puncturing of a biological tissue can be accurately performed.

In addition, according to the puncture apparatus 1, implanting of the implant 9 can be dealt with by only a minimally invasive procedure such as puncturing with the puncture member 3A of the puncture apparatus 1, without need for highly invasive incision or the like. Consequently, the burden on the patient is slight, and safety for the patient is relatively high. In addition, issues encountered in the case of conventional incision of vagina can be prevented, such as the implant 9 being exposed to the inside of the vagina via a wound formed by the incision, or complications due to infection via the wound. Thus, the safety of the operation with the puncture apparatus 1 is relatively high, and the implant 9 can be reliably implanted.

Furthermore, since the implant main body 91 can be embedded parallel to the urethra 1300, the urethra 1300 can be supported in a wider region.

In accordance with an exemplary embodiment, while the sheath 30 and the puncture needle 11 are inserted into the restriction member 13 in an assembled state as disclosed above in the series of procedure illustrated in FIGS. 14 to 16, this configuration is not restrictive. For example, inserting only the sheath 30 into the restriction member 13 and simply puncturing with the sheath 30 may also be adopted. In this case, for example, the sheath 30 can be advanced to a point where the curved portion 314 crosses the obturator foramen 1102, the puncture needle 11 can be inserted into the sheath 30, and the puncturing operation advanced. In accordance with an exemplary embodiment, the distal end of the sheath 30 is preferably so structured as to be able to puncture a living body.

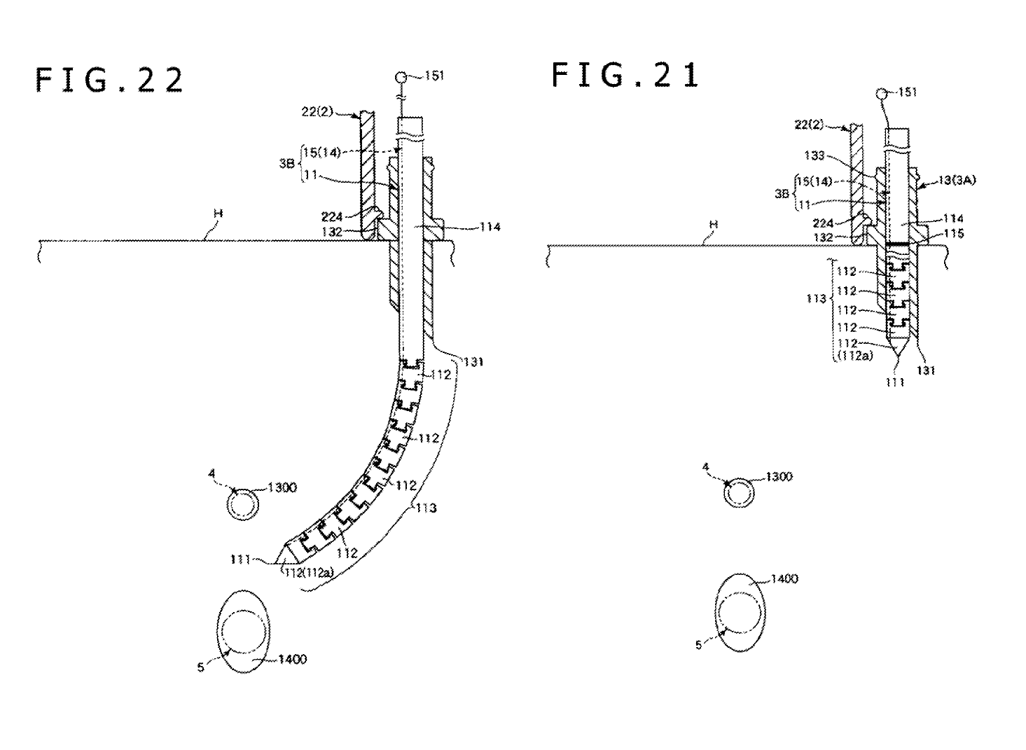

FIGS. 21 and 22 are views illustrating an operation procedure of the puncture apparatus in accordance with a second exemplary embodiment of the present disclosure. The following description will center on differences from the above-described embodiment, and descriptions of the same items as those mentioned above will be omitted. This exemplary embodiment is the same as the first embodiment above, except for a difference in a configuration of a puncture member.

As shown in FIGS. 21 and 22, in a puncture member 3B, a puncture needle 11 is composed essentially of a small piece interlock portion 113 having a plurality of small pieces 112 interlocked with one another, and a linear portion 114 extending proximally from the small piece interlock portion 113.

In the small piece interlock portion 113, the adjacent small pieces 112 are interlocked with each other by way of a recess and a projection. The small piece interlock portion 113 is rectilinearly shaped in a natural state (see FIG. 21), and is elastically deformed when an external force is exerted on interlock portion 113 (see FIG. 22). In addition, the small piece 112a located at the most distal position is provided with a sharp needle tip 111.

In accordance with an exemplary embodiment, the linear portion 114 is higher than the small piece interlock portion 113 in rigidity.

The puncture member 3B can be provided with an operating wire 15 as an operating portion deforming the puncture needle 11 into a curved shape. The operating wire 15 is inserted in and passed through the puncture needle 11, and the distal end of the operating wire 15 is connected to the small piece 112a of the puncture needle 11. In addition, the operating wire 15 is provided at its proximal portion with a grasping portion 151 to be grasped at the time of operating the operating wire 15. With the grasping portion 151 grasped and with the operating wire 15 (puncture needle 11) pulled proximally, the puncture needle 11 is deformed into a curved shape. The curvature of the puncture needle 11 can vary in proportion to an amount by which the operating wire 15 is pulled. Thus, the operating wire 15 can function as a curvature-changing unit 14 changing the curvature of the puncture needle 11.

In this exemplary embodiment, the puncture member 3B can perform a first puncture and a second puncture. As shown in FIG. 21, at the time of performing the first puncture, the puncture needle 11 is in a rectilinear state under restriction by a restriction member 13, which helps permit the first puncture to be carried out. As shown in FIG. 22, at the time of performing the second puncture, a pulling operation using the operating wire 15 is conducted in a condition where the restriction by the restriction member 13 has been lifted, whereby the puncture needle 11 is deformed into a curved state, which can allow the second puncture to be reliably carried out, and, accordingly, a region between a urethra 1300 and a vagina 1400 can be punctured. With the operating wire 15 loosened after the second puncture, the puncture needle 11 returns into the rectilinear shape, so that the first puncture can be performed.

The puncture needle 11 can have a marker 115 given to the linear portion 114. With the operating wire 15 operated when the marker 115 overlaps with a body surface H, the puncture needle 11 is deformed into the curved shape, enabling transition to the second puncture.

Thus, in this exemplary embodiment, also, puncturing of a living body can accurately be carried out, whereby a puncture hole equivalent to that in the first embodiment disclosed above can be formed in the living body.

FIG. 23 is a side view showing a third exemplary embodiment of the puncture apparatus according to the present disclosure. The following description will center on differences from the above-described embodiments, and descriptions of the same items as those mentioned above will be omitted. This exemplary embodiment is the same as the second exemplary embodiment above, except for a difference in a configuration of a puncture member.

As shown in FIG. 23, a puncture member 3C is provided with a lock mechanism 16 maintaining a curved state of a puncture needle 11. The lock mechanism 16 is a mechanism which bears part of a curvature-changing unit 14, and has a helical compression spring 161. The helical compression spring 161 can be disposed in a compressed state between a flange portion 116 provided at a proximal portion of the puncture needle 11 and a flange portion 152 provided at a proximal portion of an operating wire 15.

At a time of performing a first puncture, the flange portion 152 of the operating wire 15 is pushed in a direction compressing the helical compression spring 161, thereby conducting the puncture.

In accordance with an exemplary embodiment, at a time of performing a second puncture, the push on the flange portion 152 is removed, and the puncture is conducted. When the push on the flange portion 152 is removed, the operating wire 15 is pulled proximally due to a restoring force of the helical compression spring 161, and as a result, the puncture needle 11 is put into a curved state, which curved state is maintained. With the curved state thus maintained, the second puncture can be carried out relatively easily.

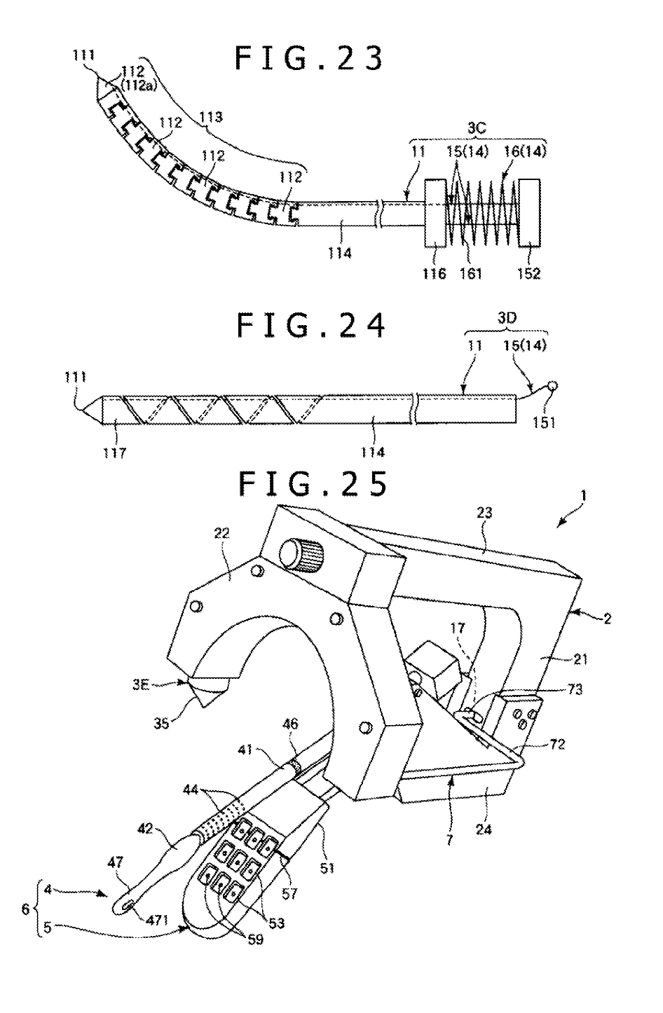

FIG. 24 is a side view showing a fourth exemplary embodiment of the puncture apparatus according to the present disclosure. The following description will center on differences from the above-described embodiments, and descriptions of the same items as those mentioned above will be omitted. This exemplary embodiment is the same as the second exemplary embodiment above, except for a difference in a configuration of a puncture member.

As shown in FIG. 24, in a puncture member 3D, a puncture needle 11 is composed essentially of a wound portion 117 having a band-shaped body (or a linear body) wound spirally, and a linear portion 114 extending proximally from the wound portion 117.

The wound portion 117 is rectilinearly shaped in a natural state (see FIG. 24). In addition, the wound portion 117 can be elastically deformed by exerting an external force on the wound portion 117, for example, by operating an operating wire 15. As a result, the curvature of the wound portion 117 is changed. Accordingly, a first puncture and a second puncture can be performed sequentially.

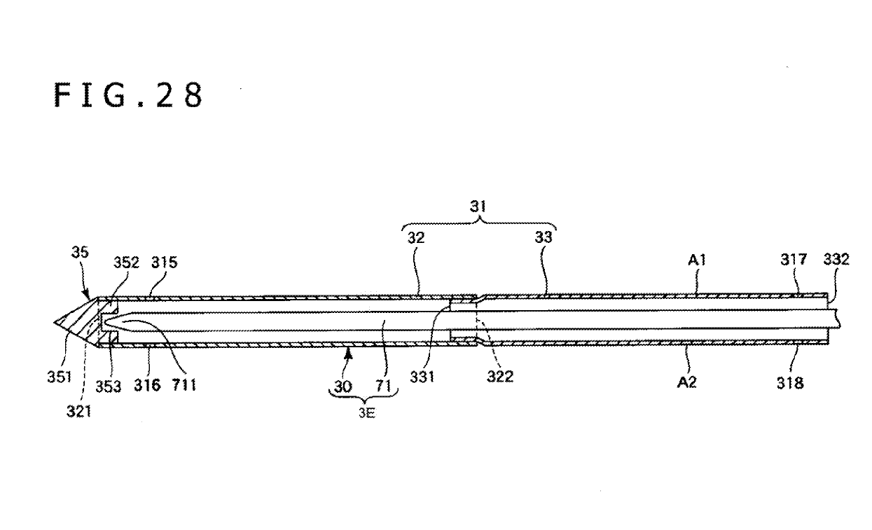

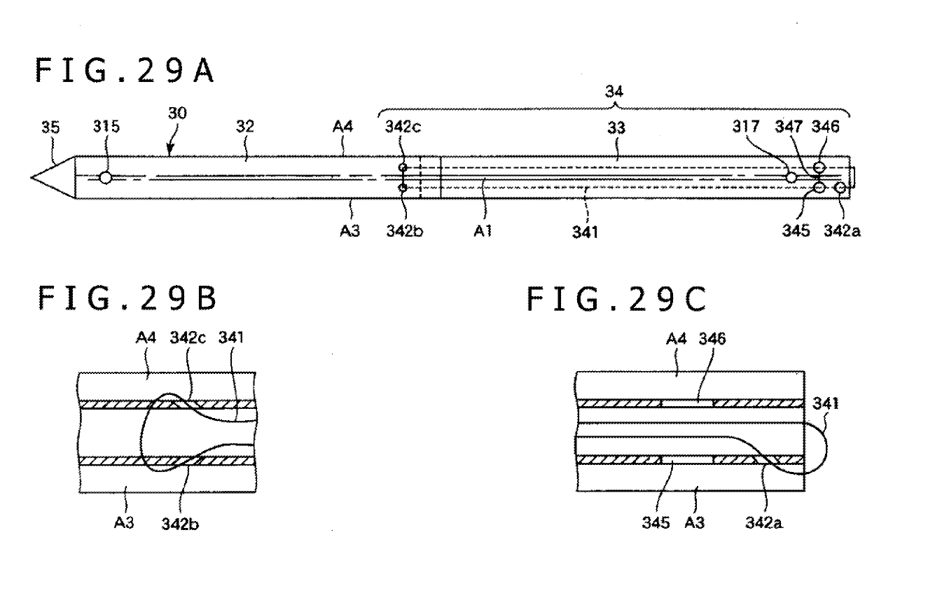

FIGS. 25-37 are views showing a fifth exemplary embodiment of the puncture apparatus according to the present disclosure. In FIGS. 28, 29A, 29B, and 29C, the puncture member actually extending in an arcuate shape is illustrated in a rectilinearly stretched state, for convenience of description. The following description will center on differences from the above-described embodiments, and descriptions of the same items as those mentioned above will be omitted. This exemplary embodiment is the same as the first exemplary embodiment above, except for differences in a configuration of a puncture apparatus.

As shown in FIG. 25, the puncture apparatus 1 can include a frame 2, a puncture member 3E, a urethral insertion member 4, a vaginal insertion member 5, and an operating member 7. The puncture member 3E, the urethral insertion member 4, and the vaginal insertion member 5 are supported on the frame 2. A main difference of the puncture apparatus 1 in this exemplary embodiment from the puncture apparatus 1 in the first embodiment above lies in the puncture member 3E.

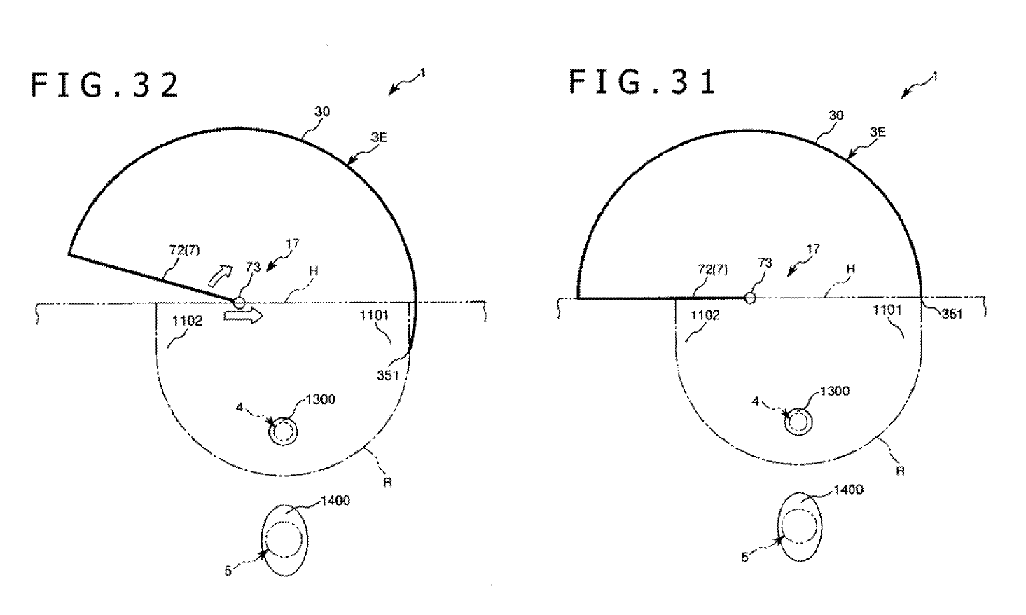

As shown in FIGS. 31 to 36, the puncture member 3E is puncturing a living body, while securing a target puncture route R, to form a puncture hole. As shown in FIG. 28, the puncture member 3E includes a sheath 30, a needle body 35 provided at a distal end of the sheath 30, and an operating member 7 (insertion portion 71) operating the sheath 30. The sheath 30 and the needle body 35 constitute a puncture needle. In addition, the sheath 30 has a tube-shaped main body 31, and a state-maintaining mechanism 34.

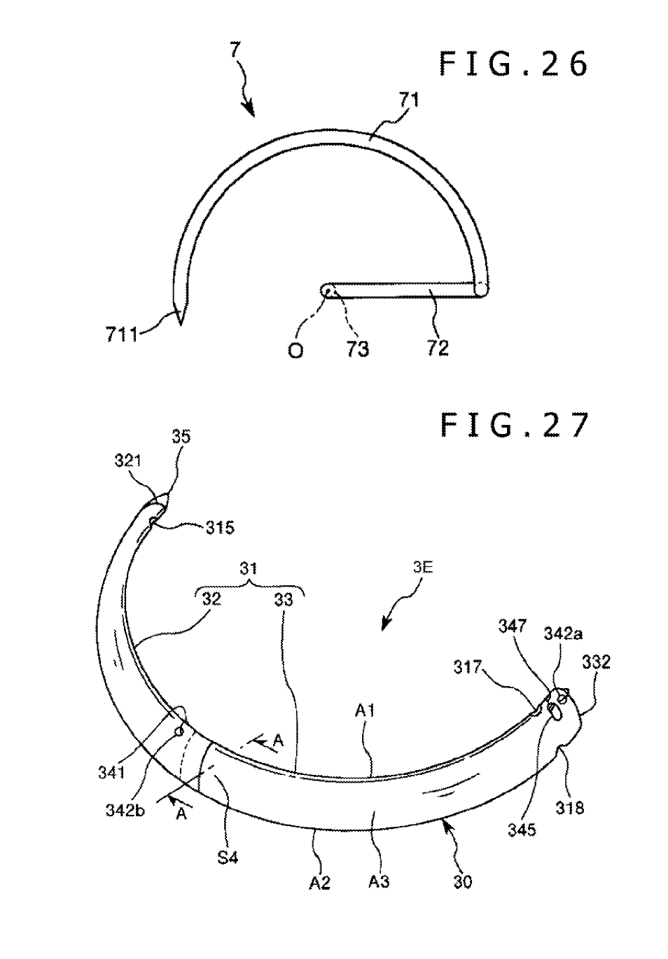

The operating member 7 is a member operating the sheath 30. Such an operating member 7, as shown in FIG. 26, can include the insertion portion 71, a shaft portion 73, and an interlock portion 72 interlocking the insertion portion 71 and the shaft portion 73 to each other. The insertion portion 71, the interlock portion 72 and the shaft portion 73 may be formed integrally; or, alternatively, at least one of the portions may be formed as a separate body in relation to the other portions.

The insertion portion 71 is a portion to be inserted in the sheath 30, and functions as a stylet that supports the sheath 30 from the inside. With the insertion portion 71 inserted in the sheath 30, the sheath 30 can be interlocked to the operating member 7, whereby it is enabled to turn the sheath 30 by the operating member 7. Such an insertion portion 71 is in an arcuate shape corresponding to the shape of the sheath 30. A center angle of the insertion portion 71 is set in conformity with a center angle of the sheath 30. A distal portion 711 of the insertion portion 71 can be tapered off. In accordance with an exemplary embodiment, the presence of the tapered distal portion 711 can enable a smooth insertion of the insertion portion 71 into the sheath 30.

The shaft portion 73 is a turning shaft which intersects a center O of the insertion portion 71 and serves as a center of turn of the sheath 30 (needle body 35). The shaft portion 73 extends along an axis J1 orthogonal to a plane f1 that contains the insertion portion 71.

The interlock portion 72 interlocks a proximal portion of the insertion portion 71 and a distal portion of the shaft portion 73 to each other. In addition, the interlock portion 72 is substantially L-shaped, with a substantially rectangular bend at an intermediate portion of the L-shaped interlock portion 72. The interlock portion 72 functions also as a grasping portion to be grasped by an operator at the time of operating the operating member 7.

Such an operating member 7 can be configured to be higher than the sheath 30 in rigidity. The material constituting the operating member 7 is not restricted. Examples of the material applicable here include various metallic materials such as stainless steels, aluminum, aluminum alloys, titanium, and titanium alloys.

As shown in FIGS. 27 and 28, in this exemplary embodiment, a main body 31 of the sheath 30 can be curved in a bow-like shape, can be rigid, and can be composed of two split pieces so that it can be split at an intermediate portion of the main body 31. For example, the main body 31 can be divided into a distal split piece 32 and a proximal split piece 33. The distal split piece 32 and the proximal split piece 33 can be approximately equal in length, and their boundary is located at a central portion S4.

As shown in FIG. 28, the distal split piece 32 can be tubular in shape, and can have a distal end opening 321 and a proximal end opening 322. Similarly, the proximal split piece 33 can be tubular in shape, and can have a distal end opening 331 and a proximal end opening 332. A distal portion of the proximal split piece 33 can be inserted into a proximal portion of the distal split piece 32, whereby the distal split piece 32 and the proximal split piece 33 are connected to each other. With the proximal split piece 33 thus inserted into the distal split piece 32, a step which can be generated at a boundary between the split pieces 32 and 33 is insusceptible to be caught on a biological tissue, so that puncturing of a living body by the puncture member 3E can be performed relatively smoothly. In accordance with an exemplary embodiment, the distal split piece 32 may be inserted into the proximal split piece 33 to connect the split pieces 32 and 33 to each other.

The material constituting the main body 31 can be, for example, a rigid material that can maintain the shape and an internal space of main body 31 in the state of being inserted in a body. Examples of such rigid material applicable can include various resin materials such as polyethylene, polyimides, polyamides, polyester elastomers, polypropylene, etc. and various metallic materials such as stainless steels, aluminum, aluminum alloys, titanium, and titanium alloys. In accordance with an exemplary embodiment, instead of adopting a rigid material constituting the main body 31, other materials may also be adopted, in which case a wall of the main body 31 is reinforced with a reinforcement member, whereby the preferable properties as above-mentioned are attained. For instance, a high-strength braiding may be embedded in the wall of the main body 31, whereby the shape and the internal space of the main body 31, in the state of being inserted in a body, can be maintained. Another example of the reinforcement member is a spiral body, which may be embedded in the wall of the main body 31, whereby the main body 31 can have flexibility while retaining the internal space to such an extent that an inserted body can be slid therein.

In accordance with an exemplary embodiment, the connected state of the split pieces 32 and 33 thus connected can be maintained by the state-maintaining mechanism 34. As shown in FIG. 29A, the state-maintaining mechanism 34 can include holes 342a, 342b, and 342c, an endless string (interlock member) 341 passed through the holes 342a, 342b, and 342c, exposure holes (through-holes) 345 and 346 exposing the string 341, and a slit 347 joining the exposure holes 345 and 346.

The hole 342a can be provided in a proximal portion of the proximal split piece 33, for example, at a front surface A3 near an inner circumferential portion A1. The holes 342b and 342c can be oppositely provided in a proximal portion of the distal split piece 32, for example, at the front surface A3 and a back surface A4 near the inner circumferential portion A1.

The string 341 is disposed inside the main body 31. The string 341 can be exposed to the outside of the main body 31, between the hole 342b and the hole 342c, and between the hole 342a and the proximal end opening 332. With the string 341 laid around in this manner, the connected state of the split pieces 32 and 33 can be securely maintained. In addition, the exposure of the string 341 to the outside of the main body 31 can be suppressed, so that the string 341 is less liable to be caught on a biological tissue. In addition, while making it possible to cut the string 341 as will be described later, an overall length of the string 341 can be made as short as possible. Therefore, at a time of inserting and passing an implant main body 91 into and through the main body 31 of the sheath 30, the string 341 is less liable to be caught on the implant main body 91. In addition, since the holes 342a, 342b, and 342c are disposed near the inner circumferential portion A1 as above-mentioned, the string 341 is also disposed near the inner circumferential portion A1, which can ensure that at the time of inserting the implant main body 91 into the main body 31 of the sheath 30, the string 341 is less liable to be caught on the implant main body 91.

The string 341 can be obtained, for example, in the following manner. A string having ends is prepared. One end of the string is inserted through the proximal end opening 332 into the inside of the main body 31, is drawn out via the hole 342b to the outside of the main body 31, is inserted through the hole 342c into the inside of the main body 31, is drawn out via the hole 342a to the outside of the main body 31, and is finally tied with the other end of the string near the proximal end opening 332. The position of the knot is not restricted.

As shown in FIG. 29C, an axis of the hole 342a is so inclined that an outside opening of the hole 342a is located on the proximal end of an inside opening of the hole 342a. As shown in FIG. 29B, an axis of each of the holes 342b and 342c can be so inclined that an outside opening of the hole is locate on the distal end of an inside opening of the hole, which can enable the holes 342a, 342b, and 342c to extend along the course of the string 341, so that the string 341 is less liable to be caught on the holes 342a, 342b, and 342c.

The exposure holes 345 and 345 can be oppositely provided in the front surface A3 and in the back surface A4, at a proximal portion of the proximal split piece 33. The part where the exposure holes 345 and 346 are provided protrudes from a body surface H, in the condition where the main body 31 is disposed in a body. Therefore, the string 341 is exposed via the exposure holes 345 and 346 to the outside of the main body 31. In addition, the exposure holes 345 and 346 are joined by the slit 347 which is provided in the inner circumferential portion A1 along a circumferential direction of the main body 31.

In the state-maintaining mechanism 34 as disclosed above, cutting the string 341 can result in a state wherein the distal split piece 32 and the proximal split piece 33 can be separated from each other. This configuration can ensure that the distal split piece 32 and the proximal split piece 33 can be put into a separable state by a simple operation. In addition, since the cutting of the string 341 can be visually checked, it can be easily confirmed that the distal split piece 32 and the proximal split piece 33 have been put into the separable state.

The exposure holes 345 and 346 and the slit 347 thus provided, as in this exemplary embodiment, enable easy cutting of the string 341. For example, a pair of scissors having a pair of cutting blades (a first blade and a second blade) can be prepared. The first blade is inserted and passed in the exposure holes 345 and 346, and the string 341 is located between the pair of blades. Then, the scissors is put into a closing operation, whereon at least one of the first and second blades passes the slit 347, and the first and second blades overlap each other. During this process, the string 341 is cut. In this way, the exposure holes 345 and 346 and the slit 347 provided as above-mentioned enable easy cutting of the string 341.

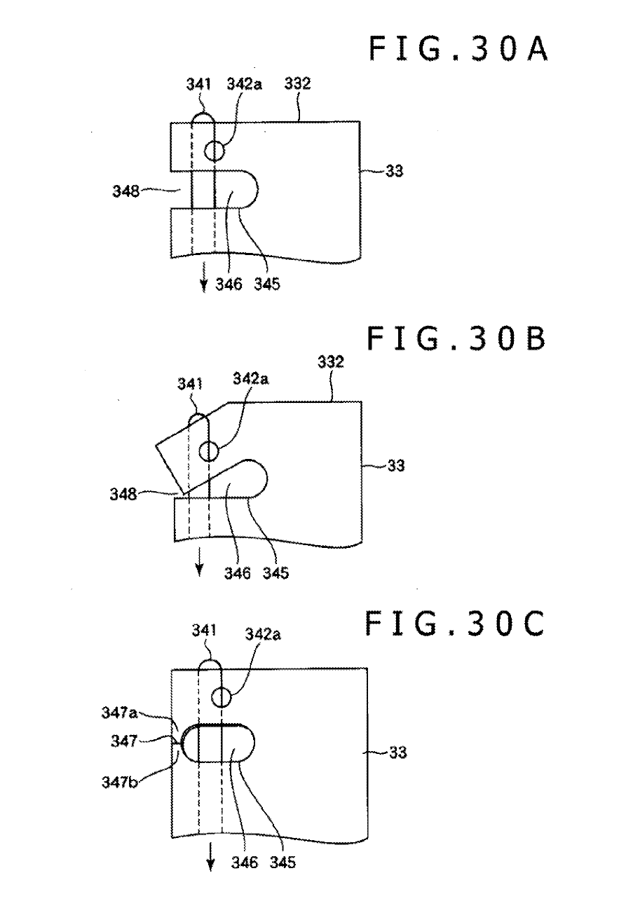

Thus, in this exemplary embodiment, the slit 347 is provided, and the slit 347 is used as a passing route for the blade, which can help ensure that deformation of the main body 31 due to a tension on the string 341 is relatively prevented. For example, as shown in FIG. 30A, the passing route for the blade may be configured by use of a hole 348 in place of the slit 347. In this case, however, the hole 348 may, depending on the hardness of the main body 31 or the like factor, be collapsed through buckling under the tension on the string 341, and the main body 31 may be deformed thereby, as shown in FIG. 30B. In the case of the slit 347, those parts 347a and 347b on opposite sides of the slit 347 abut against each other, so that the above-mentioned deformation would not occur, and deformation of the main body 31 is relatively prevented, as shown in FIG. 30C.

The number and layout of the holes (342a, 342b, 342c) through which to pass the string 341 are not limited, insofar as the connected state of the distal split piece 32 and the proximal split piece 33 can be maintained by the string 341. In addition, the string 341 may not necessarily be in an endless form, but may have ends, namely, one end and the other end. For instance, a string having ends may be prepared, one end of the string may be passed through the hole 342a and the proximal end opening 332 and be looped, and the other end of the string may be passed through the holes 342b and 342c and be looped. Furthermore, the string 341 can include braiding, bands and the like that can be used in the same manner as the string 341.

In addition, as shown in FIG. 28, the main body 31 is provided at its distal portion with a pair of engaging holes 315 and 316 to be used for other purpose, for example, facilitating the operation of the sheath 30. Similarly, the main body 31 is provided at its proximal portion with a pair of engaging holes 317 and 318 to be used for other purpose. Of these four engaging holes, the engaging holes 315 and 317 are provided in the inner circumferential portion A1, whereas the engaging holes 316 and 318 are provided in the outer circumferential portion A2.

Besides, a spacing between the engaging holes 315 and 316 and the central portion S4 and a spacing between the engaging holes 317 and 318 and the central portion S4 are approximately equal, which can help ensure that the position of the central portion S4 of the main body 31 in a living body can be easily grasped.

The main body 31 as above is provided with the needle body 35 at the distal end of the main body 31. As shown in FIG. 28, the needle body 35 has a needle tip 351 which is tapered off, and a proximal portion 352 provided on the proximal end of the needle tip 351. The proximal portion 352 is inserted in the main body 31, whereby the needle body 35 is held inside the main body 31 in a freely detachable manner. The proximal portion 352 is fitted in the main body 31 with such a force that unintended detachment of the needle body 35 from the main body 31 can be relatively prevented. Further, the needle body 35 may be configured integrally with the main body 31.

In addition, the proximal portion 352 can be provided with an engaging portion 353 for engagement with the distal portion 711 of the insertion portion 71. The engaging portion 353 can be composed essentially of a recess. In an inserted state wherein the puncture member 3E is inserted in the insertion portion 71, the distal portion 711 is located inside the engaging portion 353. The engaging portion 353 thus provided ensures that displacement of the needle body 35 relative to the insertion portion 71 is suppressed, and puncturing of a living body by the puncture member 3E can be carried out smoothly.

In the case where at least the distal portion 711, of the insertion portion 71, is flat-shaped in cross section, for example, the cross-sectional shape of the engaging portion 353 is set in conformity with the cross-sectional shape of the distal portion 711, for example, the engaging portion 353 is also flat-shaped in cross section, which helps ensure that when the engaging portion 353 and the distal portion 711 are in engagement with each other, the flat shape of the engaging portion 353 and the flat shape of the distal portion 711 overlap each other. This overlap can help ensure that the sheath 30 is restrained from rotating relative to the insertion portion 71, about the axis of the insertion portion 71, for example, about an axis extending along the puncturing direction. Consequently, a curved puncture route which reliably passes between the urethra 1300 and the vagina 1400 can be secured.

The material constituting the needle body 35 is not restricted. For example, the same materials as those for the main body 31 can be used for the needle body 35.

As shown in FIG. 25, a support unit 17 supporting the puncture member 3E (operating member 7) is incorporated and disposed in a grasping portion 21 of the frame 2. The support unit 17 can support the puncture member 3E in such a manner that the puncture member 3E can be turned about the shaft portion 73. In addition, the support unit 17 can support the puncture member 3E also in such a manner that the puncture member 3E, while being turned about the shaft portion 73, can be moved in a direction orthogonal to the shaft portion 73, for example, in a horizontal direction in a use state (mounted state) of the puncture apparatus 1 in this exemplary embodiment.

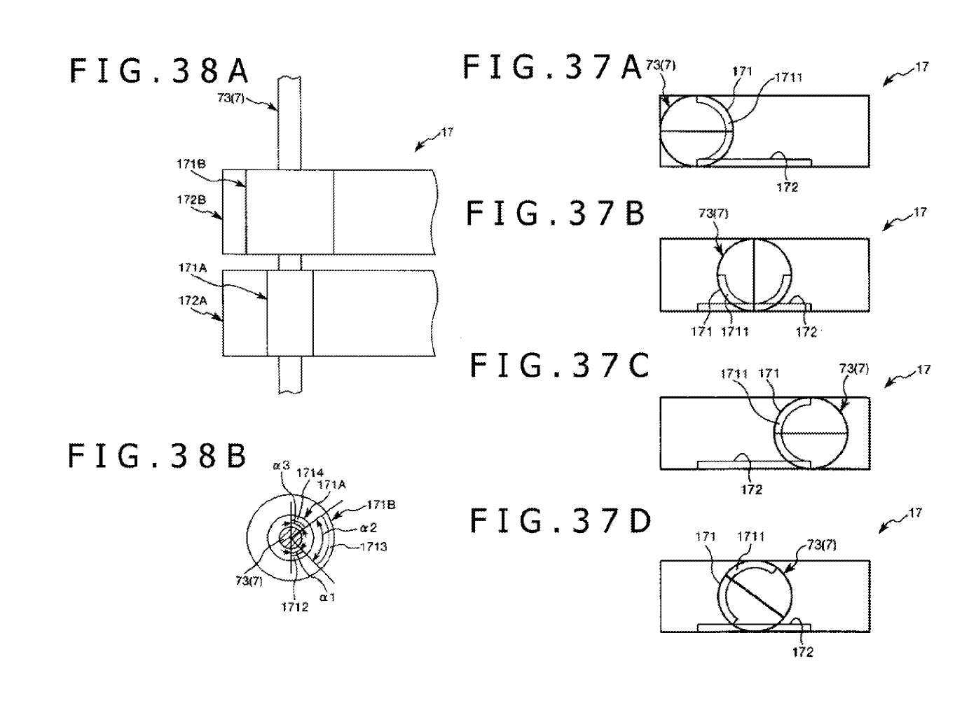

As shown in FIGS. 37A to 37D, the support unit 17 can include a pinion gear 171 which is fixedly provided on the shaft portion 73 and rotated together with the shaft portion 73, and a rack 172 which is fixedly provided on the grasping portion 21 and meshes with the pinion gear 171.

The pinion gear 171 has teeth 1711 formed along the circumferential direction of the shaft portion 73, in a region of half the circumference. The rack 172 is so formed that an overall length of its toothed portion is equal to an overall length of the teeth 1711 of the pinion gear 171, which can ensure that the support unit 17 can assume a state in which the pinion gear 171 is in mesh with the rack 172, as shown in FIGS. 37A to 37C, and a state in which the pinion gear 171 is out of mesh with the rack 172 and can be moved in the left-right direction in the figure while rotating idly, as shown in FIG. 37D.

By the support unit 17 configured in this way, the puncture member 3E is operated so as to sequentially perform a first puncture (a puncture depicted in FIG. 31 through FIG. 32), a second puncture (a puncture depicted in FIG. 32 through FIG. 35), and a first puncture (a puncture depicted in FIG. 36). This can help ensure that the orbital path described by the needle tip 351 of the puncture member 3E is a target puncture route R, and, accordingly, the puncture member 3E punctures a living body along the target puncture route R.

A method of using the puncture apparatus 1 (operation procedure) in this exemplary embodiment will be described below, referring to FIGS. 31 to 37D.

As shown in FIG. 31, the puncture apparatus 1 is set into the mounted state, and the needle tip 351 is set in abutment on a body surface H at the right-hand inguinal region of a patient or a region in the vicinity thereof (first region). In this instance, the support unit 17 is in a state as shown in FIG. 37A.

Next, while grasping an interlock portion 23 of the frame 2 by one hand, for example, an interlock portion 72 of the operating member 7 is grasped by the other hand, and the operating member 7 is rotated clockwise, as shown in FIG. 32. This can help ensure that the needle tip 351 of the puncture member 3E punctures the body surface H at the right-hand inguinal region of the patient or the region in the vicinity thereof (first region) by the first puncture, to enter the body. This first puncture is performed until the needle tip 351 reaches an obturator foramen 1101.

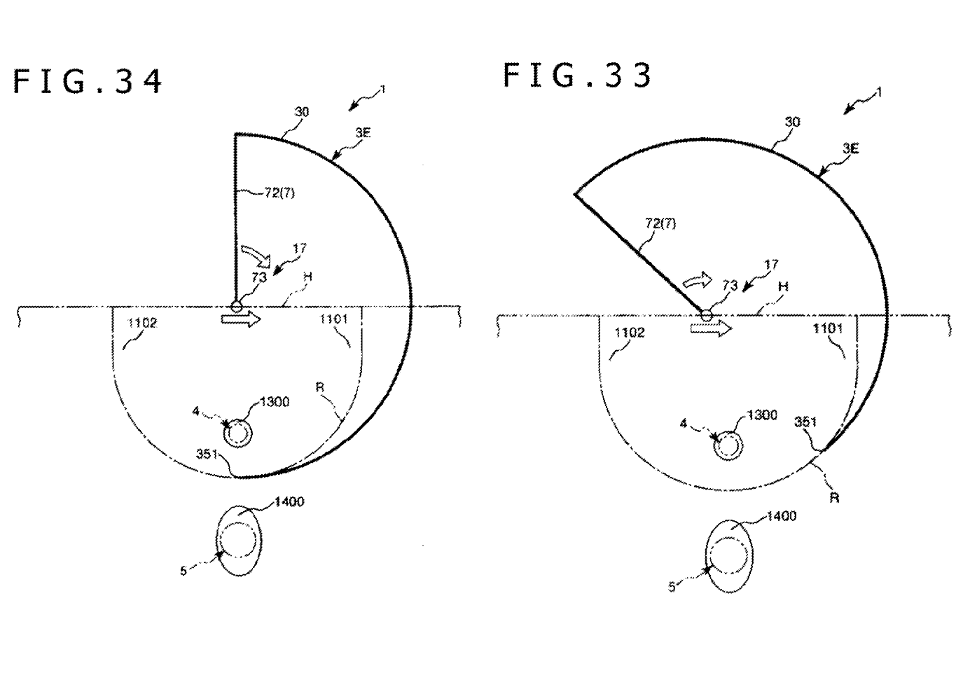

As the rotating operation on the operating member 7 is further continued, as shown in FIG. 33, a transition to the second puncture takes place, and puncturing by the second puncture is started.

Then, when a turning angle of the operating member 7 as measured from the state shown in FIG. 31 reaches 90 degrees, as shown in FIG. 34, the needle tip 351 crosses a region between the urethra 1300 and the vagina 1400. For example, in this instance, the support unit 17 is in a state as shown in FIG. 37B.

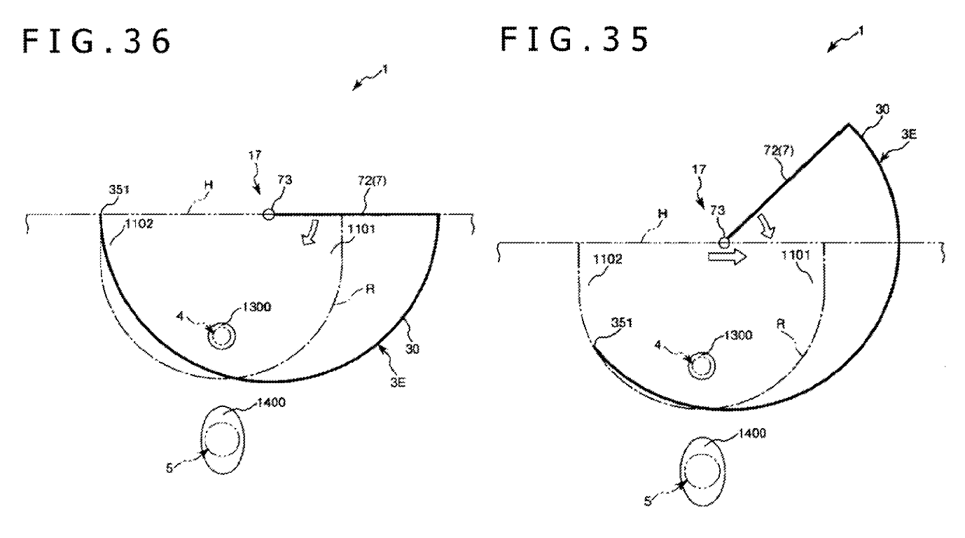

Thereafter, the living body can be punctured by the second puncture until immediately before the needle tip 351 passes an obturator foramen 1102, as shown in FIG. 35.

When the turning angle of the operating member 7 as measured from the state shown in FIG. 31 reaches 180 degrees, as shown in FIG. 36, the needle tip 351 has passed the obturator foramen 1102 and reaches a position immediately beneath the body surface H at the left-hand inguinal region or a region in the vicinity of the body surface H at the left-hand inguinal region. For example, in this instance, the support unit 17 is in a state as shown in FIG. 37C.

As the rotating operation on the operating member 7 is further continued, as shown in FIG. 37D, the pinion gear 171 is disengaged from the rack 172, and the shaft portion 73 is rotated idly, which results in that the needle tip 351 protrudes from the body surface H to the outside of the body.

By the puncturing operation as above, a puncture hole in a bow-like shape can be formed in the living body proximate to the target puncture route R. This puncture needle also is in the state of reliably passing between the urethra 1300 and the vagina 1400, like that in the first exemplary embodiment described above. Thus, the puncture apparatus 1 in this exemplary embodiment also can help ensure that puncturing of a biological tissue can be accurately carried out.

Next, the needle body 35 is detached from the sheath 30, and the operating member 7 is rotated in a direction reverse to the aforementioned. This results in that the inserted portion 71 of the operating member 7 is pulled out of the sheath 30, with the sheath 30 left in the living body.

Subsequently, the implant main body 91 is inserted into the sheath 30 while being taken out of a wrapping material 92, and a band 912 is set into a state of protruding from the proximal end opening and the distal end opening of the sheath 30.

Next, that portion of the string 341 which is exposed from the exposure holes 345 and 346 is cut. This results in a state in which the sheath 30 can be split into the distal split piece 32 and the proximal split piece 33. The exposure holes 345 and 346 are located so as to be exposed to the outside of the living body, so that the cutting of the string 341 can be easily carried out.

Subsequently, the connection between the distal split piece 32 and the proximal split piece 33 is canceled, then the distal split piece 32 is pulled distally out of the living body, and the proximal split piece 33 is pulled proximally out of the living body. For example, in this case, the distal split piece 32 and the proximal split piece 33 are moved in the opposite directions substantially simultaneously, and the distal split piece 32 and the proximal split piece 33 are moved along an arcuate path such as to follow their own shape, which can permit the sheath 30 to be smoothly removed from the living body. As the distal split piece 32 and the proximal split piece 33 are gradually taken out of the living body as above-mentioned, the surrounding tissue having been forced open (expanded) by the sheath 30 returns into an original position, and the tissue comes into contact with the implant main body 91, gradually from a central portion toward both end portions of the implant main body 91. As above-mentioned, the distal split piece 32 and the proximal split piece 33 are moved in directions along their shapes, and the sheath 30 has an internal space such that the implant main body 91 can be moved therein under a sufficiently low friction, which can help ensure that the implant main body 91 can be placed indwelling in an as-is state, with no unnecessary tensile force exerted on the implant main body 91. Accordingly, there is no need for control of tension on the implant main body 91. As a result of these operations, the implant main body 91 is in the state of being embedded in the living body.

In addition, with the sheath 30 thus split into the split pieces and removed from the living body as the split pieces, pulling-out of the sheath 30 from the living body can be carried out rather easily. In addition, the pulling-out method can help ensure that the split pieces 32 and 33 being pulled out exert little influence on a posture of the main body portion 911 in the region between the urethra 1300 and the vagina 1400.

FIGS. 38A and 38B illustrate a support unit possessed by the puncture apparatus in accordance with a sixth exemplary embodiment of the present disclosure, wherein FIG. 38A is a plan view and FIG. 38B is a cross-sectional view. The following description will center on differences from the above-described embodiments, and descriptions of the same items as those mentioned above will be omitted. This exemplary embodiment is the same as the fifth exemplary embodiment above, except for a difference in a configuration of a support unit supporting a puncture member.

As shown in FIGS. 38A and 38B, in this exemplary embodiment, a support unit 17 can include a first pinion gear 171A and a second pinion gear 171B which are fixedly provided on a shaft portion 73 of an operating member 7; and a first rack 172A and a second rack 172B which are fixedly provided on a grasping portion 21 of a frame 2.

As shown in FIG. 38A, the first pinion gear 171A and the second pinion gear 171B are disposed adjacently along the shaft portion 73. In addition, the first rack 172A is disposed so as to mesh with the first pinion gear 171A, and the second rack 172B is disposed so as to mesh with the second pinion gear 171B.

As shown in FIG. 38B, the first pinion gear 171A has teeth 1712 and 1714, and the second pinion gear 171B has teeth 1713.

In the support unit 17, when the shaft portion 73 is rotated, the teeth 1712, 1713, and 1714 are sequentially put into mesh in this order, attendant on the rotation. For example, when the shaft portion 73 is rotated, first, the teeth 1712 of the first pinion gear 171A meshes with the first rack 172A and comes out of mesh, during when the first pinion gear 171A is rotated by a predetermined angle α1 (for example, 45 degrees). Next, the teeth 1713 of the second pinion gear 171B meshes with the second rack 172B and comes out of mesh, during when the second pinion gear 171B is rotated by a predetermined angle α2 (for example, 90 degrees). Subsequently, the teeth 1714 of the first pinion gear 171A meshes with the first rack 172A and comes out of mesh, during when the first pinion gear 171A is rotated by a predetermined angle α3 (for example, 45 degrees).

In addition, a gear ratio between the first pinion gear 171A and the first rack 172A and a gear ratio between the second pinion gear 171B and the second rack 172B are different from each other. For example, the former gear ratio is lower than the latter gear ratio.

When a first puncture (to be conducted first) is conducted, by use of the support unit 17 configured as above, the teeth 1712 of the first pinion gear 171A meshes with the first rack 172A. When a second puncture is performed, the teeth 1713 of the second pinion gear 171B meshes with the second rack 172B. When a first puncture (to be conducted last) is conducted, the teeth 1714 of the first pinion gear 171A meshes with the first rack 172A. By this process, the horizontal moving speed of the needle tip 351 can be varied. For example, the needle tip 351 is moved at a low speed in the first puncture and at a high speed in the second puncture. Accordingly, a puncture hole can be formed proximate to the target puncture route R.

The support unit 17, a configuration may be adopted in which, unlike in the configuration of FIG. 38B, the first pinion gear 171A is provided with “teeth” in a range of the predetermined angle α2, and the second pinion gear 171B is provided with “teeth” in ranges of the predetermined angles α1 and α3.

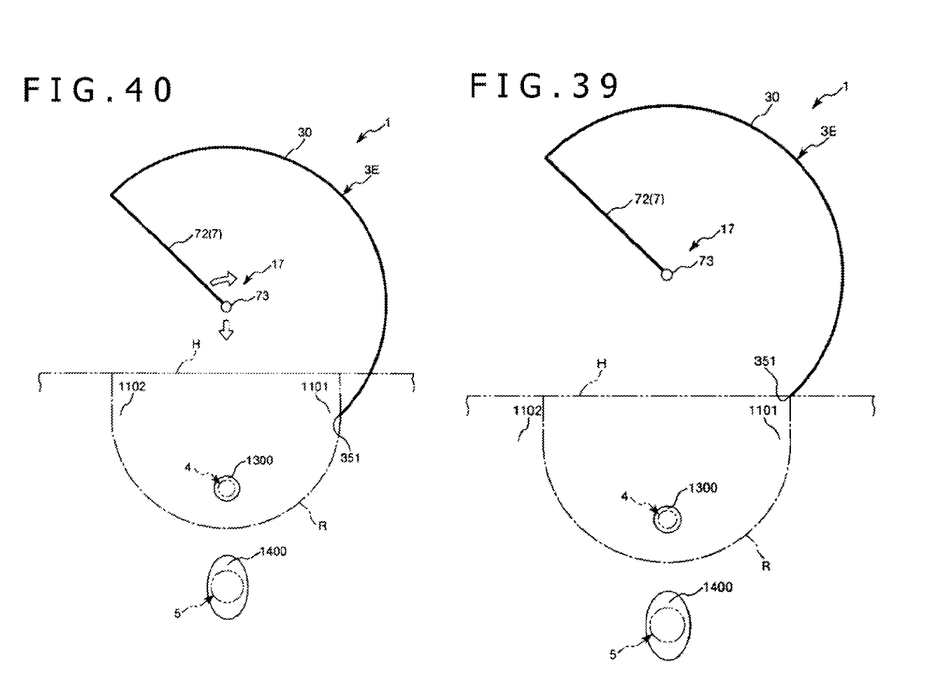

FIGS. 39-47 are views illustrating an operation procedure of the puncture apparatus in accordance with a seventh exemplary embodiment of the present disclosure. Now, referring to these figures, the seventh exemplary embodiment of the puncture apparatus and puncturing method according to the present disclosure will be described below. The following description will center on differences from the above-described embodiments, and descriptions of the same items as those mentioned above will be omitted. This exemplary embodiment is the same as the fifth exemplary embodiment above, except for a difference in a configuration of a support unit supporting a puncture member.

As shown in FIGS. 39 and 40, in this exemplary embodiment, a support unit 17 can support a puncture member 3E in such a manner that the puncture member 3E, while being turned about a shaft portion 73, can be reciprocated in the vertical direction in the use state of the puncture apparatus 1.

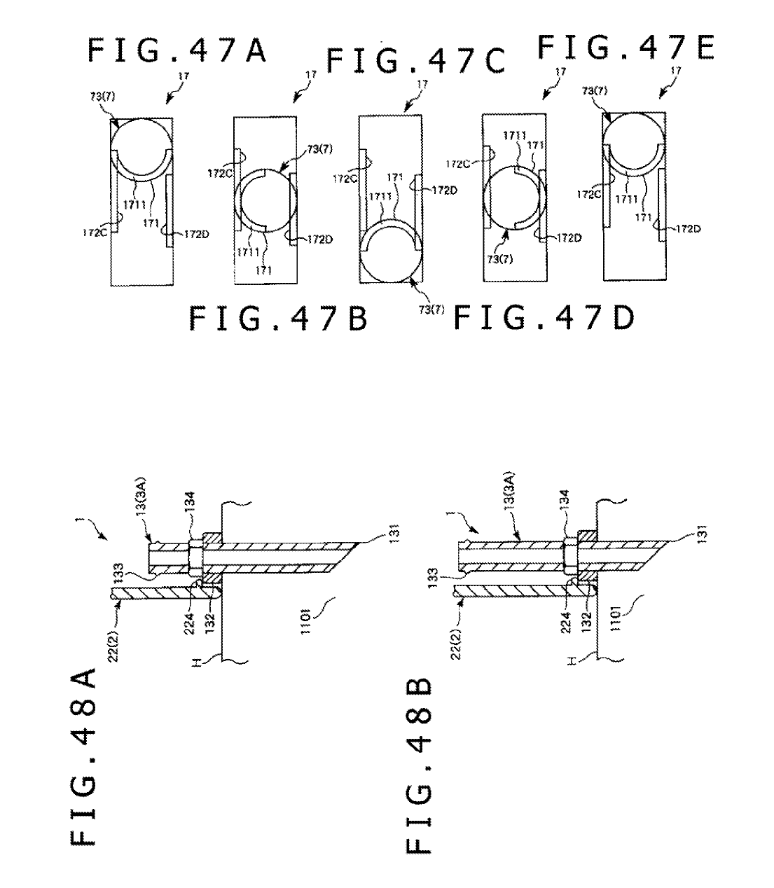

As shown in FIGS. 47A to 47E, the support unit 17 can include a pinion gear 171 which is fixedly provided on the shaft portion 73 and rotated together with the shaft portion 73 and a first rack 172C and a second rack 172D which are fixedly provided on a grasping portion 21 of a frame 2 and are disposed opposite to each other, with the pinion gear 171 interposed therebetween. The first rack 172C and the second rack 172D are disposed with slight deviation from each other along a moving direction of the pinion gear 171. In addition, each of the first rack 172C and the second rack 172D is so formed that an overall length of a toothed portion of first rack 172C and the second rack 172D is equal to an overall length of teeth 1711 of the pinion gear 171. The support unit 17 can assume a state in which the pinion gear 171 is in mesh with the first rack 172C, as shown in FIGS. 47A, 47B, and 47E, and a state in which the pinion gear 171 is in mesh with the second rack 172D, as shown in FIGS. 47C and 47D.

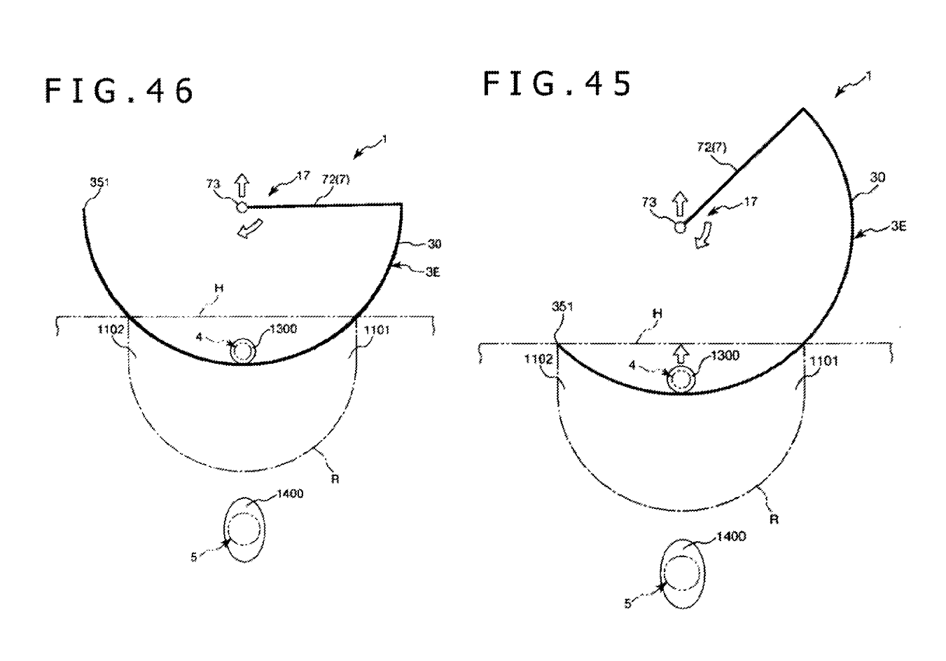

By the support unit 17 configured as above, the puncture member 3E is operated to sequentially perform a first puncture (a puncture depicted in FIG. 39 through FIG. 40), a second puncture (a puncture depicted in FIG. 40 through FIG. 44), and a first puncture (a puncture depicted in FIG. 44 through FIG. 45), which can help ensure that an orbital path described by a needle tip 351 of the puncture member 3E is a target puncture route R, and, accordingly, the puncture member 3E punctures a living body along the target puncture route R.

A method of using the puncture apparatus 1 (operation procedure) in this exemplary embodiment will be described below, referring to FIGS. 39 to 47E.

As shown in FIG. 39, the puncture apparatus 1 is set in a mounted state, and the needle tip 351 is abutted on a body surface H at a right-hand inguinal region of a patient or a region in the vicinity thereof (first region). For example, in this instance, the support unit 17 is in a state as shown in FIG. 47A.

Next, while grasping an interlock portion 23 of a frame 2 by one hand, for example, an interlock portion 72 of an operating member 7 is grasped by the other hand, and the operating member 7 is rotated clockwise, as shown in FIG. 40. This causes the needle tip 351 of the puncture member 3E to puncture the body surface H at the right-hand inguinal region of the patient or the region in the vicinity thereof (the first region), by the first puncture, thereby entering the body and reaching an obturator foramen 1101.

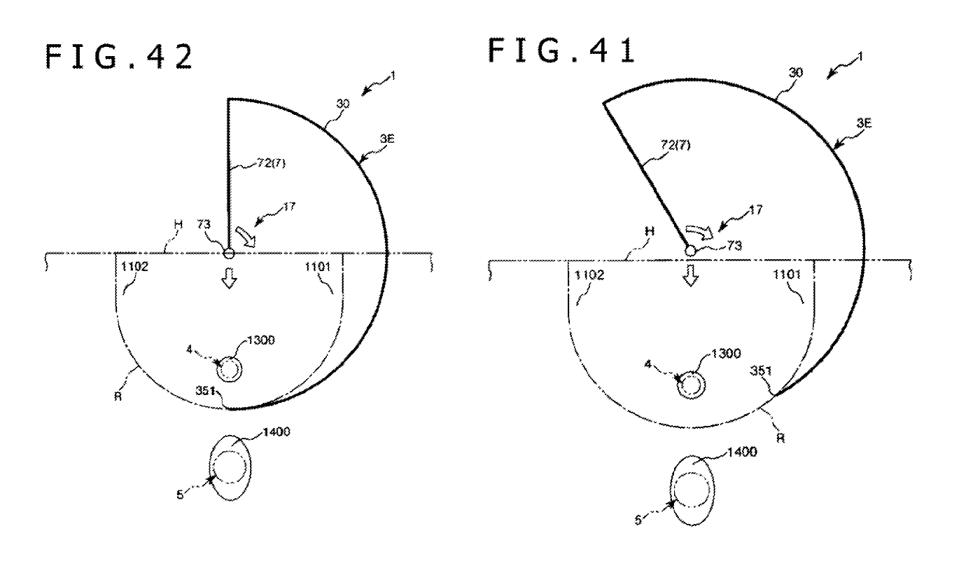

As the rotating operation on the operating member 7 is further continued, as shown in FIG. 41, transition to the second puncture takes place, and puncturing by the second puncture is conducted. For example, in this instance, the support unit 17 is in a state as shown in FIG. 47B.

Then, when a turning angle of the operating member 7 as measured from the state shown in FIG. 39 reaches 45 degrees, as shown in FIG. 42, the needle tip 351 crosses a region between a urethra 1300 and a vagina 1400. For example, in this instance, the support unit 17 is in a state as shown in FIG. 47C, for example, the pinion gear 171 comes out of mesh with the first rack 172C and meshes with the second rack 172D.

Up to this point is a puncture route that is obtained in a forward course (like an outward journey).

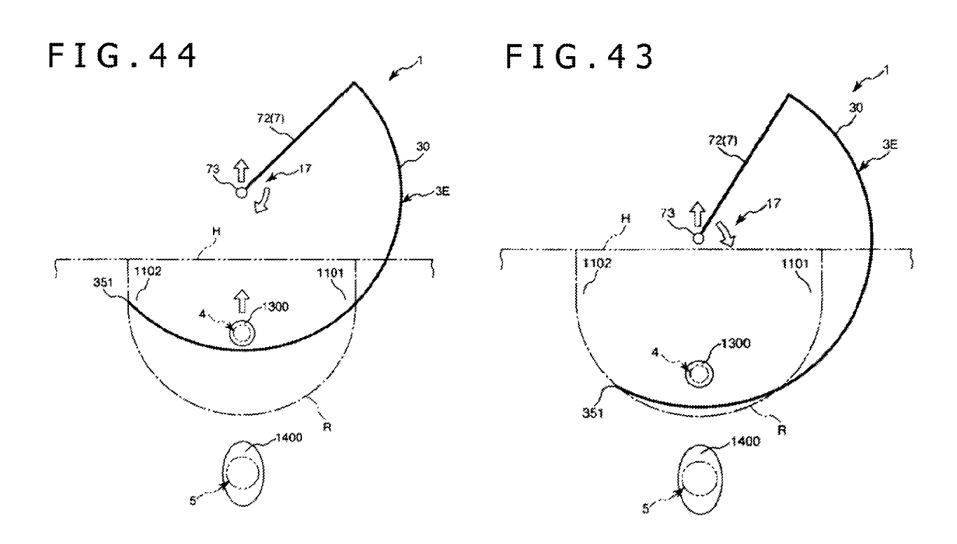

Thereafter, as the rotating operation is continued further, as shown in FIGS. 43 and 44, the living body can be punctured by the second puncture until immediately before the needle tip 351 crosses an obturator foramen 1102. For example, in this instance, the support unit 17 is in a state as shown in FIG. 47D.

The shaft portion 73 can be moved vertically upward, the puncture member 3E can also be moved in the same direction. This results in that the puncture member 3E lifts up the urethra 1300 together with a urethral insertion member 4, as shown in FIG. 44.

When the turning angle of the operating member 7 as measured from the state shown in FIG. 39 reaches 90 degrees, as shown in FIG. 45, the needle tip 351 has crossed the obturator foramen 1102, and further reaches a point immediately beneath the body surface H at a left-hand inguinal region or a region in the vicinity of the left-hand inguinal region.

Then, as the rotating operation on the operating member 7 is further continued, as shown in FIG. 46, the needle tip 351 protrudes from the body surface H to the outside of the body. For example, in this instance, the support unit 17 is in a state as shown in FIG. 47E.

Up to this point is a puncture route obtained by a return course (like a return journey).

By the puncturing operation as above, a puncture hole in a bow-like shape can be formed in the living body proximate to the target puncture route R. This puncture hole also is in the state of reliably passing between the urethra 1300 and the vagina 1400, like that in the first exemplary embodiment disclosed above. Thus, by the puncture apparatus 1 in this exemplary embodiment, also, puncturing a biological tissue can be carried out accurately.

FIGS. 48A and 48B are sectional views showing an eighth embodiment of the puncture apparatus according to the present disclosure.

Now, referring to this figure, the eighth embodiment of the puncture apparatus and puncturing method according to the present disclosure will be described below. The following description will center on differences from the above-described embodiments, and descriptions of the same items as those mentioned above will be omitted.

This exemplary embodiment is the same as the first embodiment, except for a difference in a configuration of a restriction member.

As shown in FIGS. 48A and 48B, in this exemplary embodiment, a restriction member 13 has a flange portion 132 which is so provided that it can be moved along an axial direction of the restriction member 13 and can be stopped at the moved position, through screw engagement, which can help ensure that a depth of puncture of the restriction member 13 into a living body of a patient can be regulated, thereby changing a distance of a first puncture applied to the living body, according to the body type of the patient. For instance, in a case of a somewhat large patient, the first puncture can be performed with the restriction member 13 set in a state as shown in FIG. 48A. In a case of a slim-type patient, on the other hand, the first puncture can be conducted with the restriction member 13 set in a state as shown in FIG. 48B.

While the flange portion 132 can be moved along the axial direction of the restriction member 13 and be stopped at the moved position through screw engagement as above-mentioned, for example, it can be preferable to provide a nut 134 maintaining the stopped state. After the flange portion 132 is stopped, the nut 134 is tightened from above the flange portion 132, whereby the stopped condition of the flange portion 132 can be maintained. In addition, untightening the nut 134 results in that the flange portion 132 is movable.

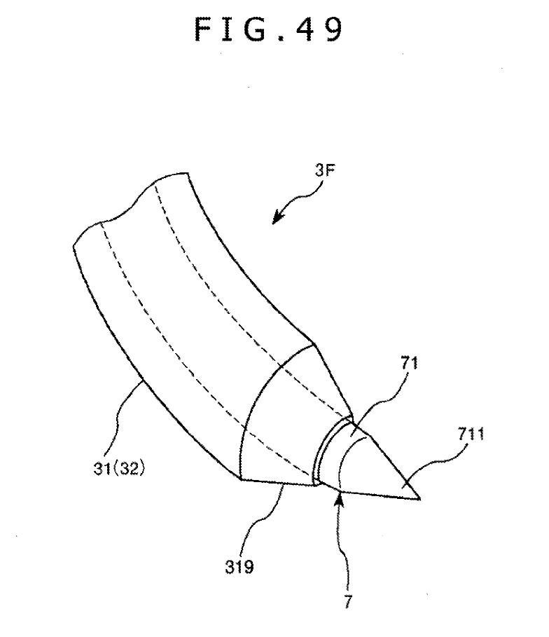

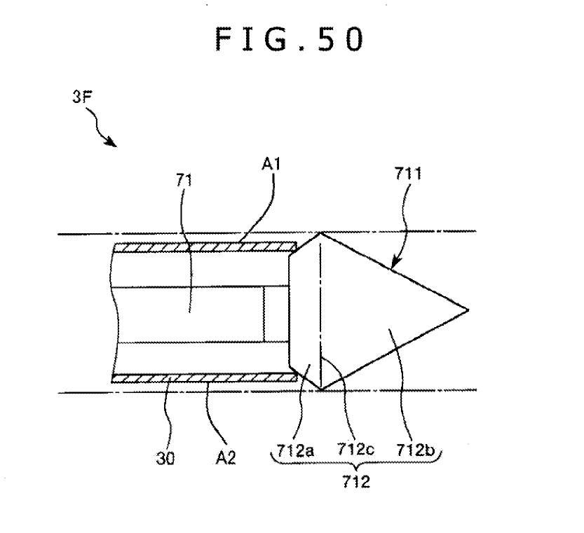

FIGS. 49 and 50 are views showing the puncture member possessed by a puncture apparatus in accordance with a ninth exemplary embodiment of the present disclosure. Now, referring to these figures, the ninth embodiment of the present disclosure will be described below. This exemplary embodiment is the same as the fifth exemplary embodiment above, except for a difference in a configuration of a puncture member.

As shown in FIG. 49, in this exemplary embodiment, there is adopted a configuration that is obtained by omitting the needle body 35 from the puncture member 3A in the fifth exemplary embodiment above. In addition, in a state (initial state) in which an insertion portion 71 is inserted in a puncture member 3F, a distal portion 711 of the insertion portion 71 is protruding from a distal end opening of a main body 31 of a sheath 30. The distal portion 711 protruding from the main body 31 functions also as a needle tip of the puncture member 3F. With the distal portion 711 of the insertion portion 71 thus serving also as the needle body of the puncture member 3F, it is enabled to reduce the number of component members, as compared with that in the above-described second embodiment, for example. In addition, when the puncture member 3F is made to puncture a living body and the insertion portion 71 is pulled out of the puncture member 3F, the distal end opening of the main body 31 can be opened. Specifically, according to this exemplary embodiment, the need for detaching the needle body 35 for opening the distal end opening of the main body 31, as in the above-described fifth embodiment, is eliminated, so that the surgical operation can be carried out smoothly. In addition, for example, since the outside diameter of the insertion portion 71 and the inside diameter of the distal end opening of the main body 31 are set to be approximately equal to each other, positional deviation of the insertion portion 71 from the main body 31 is prevented, and operability is enhanced.

In addition, the main body 31 is provided at its distal portion with a tapered portion 319 where the outside diameter gradually increases along the proximal direction from the distal end opening. The tapered portion 319 functions as a dissection portion dissecting a living body in the manner of gradually expanding the living body, following the distal portion 711, as the distal portion 711 of the insertion portion 71 punctures the living body.

A taper angle of the tapered portion 319 and a taper angle of the distal portion 711 may be equal to each other, but are preferably different from each other, as shown in FIG. 49. For example, in this case, the taper angle of the tapered portion 319 is smaller than the taper angle of the distal portion 711, which helps enable smooth puncturing.

According to the ninth exemplary embodiment as above, also, an effect equivalent to that obtained in the aforementioned fifth exemplary embodiment can be produced.

Besides, as a modification of this exemplary embodiment, the following configuration may be mentioned. As shown in FIG. 50, a puncture member 3F has a configuration obtained by omitting the needle body 35 from the puncture member 3A from the above-described fifth exemplary embodiment. In addition, in a state (initial state) in which the insertion portion 71 is inserted in the puncture member 3F, the distal portion 711 of the insertion portion 71 is protruding from a distal end opening of the main body 31.

The distal portion 711 is detachably attached to the insertion portion 71, by screw engagement, fitting or the like. The distal portion 711 has a needle tip 712 which protrudes from the distal end of the sheath 30. The needle tip 712 is in a flat shape, modeled after the sheath 30. The needle tip 712 can include a gradually increasing area portion 712a where cross-sectional area gradually increases along the distal direction; and a gradually decreasing area portion 712b where the cross-sectional area gradually decreases along the distal direction. The gradually decreasing area portion 712b is provided in the distal end of the gradually increasing area portion 712a. A minor axis of a boundary portion 712c between the gradually increasing area portion 712a and the gradually decreasing area portion 712b is longer than a minor axis of the distal end of the sheath 30, and a major axis of the boundary portion 712c is longer than a major axis of the distal end of the sheath 30. This can help ensure that the inside of a living body can be punctured substantially by only the needle tip 712. Accordingly, resistance to puncture can be reduced, and puncturing of a living body can be carried out smoothly. For example, the minor axis of the boundary portion 712c may be equal to the minor axis of the distal end of the sheath 30, and the major axis of the boundary portion 712c may be equal to the major axis of the distal end of the sheath 30.

While the puncture apparatus and puncturing method according to the present disclosure have been described above referring to the embodiments illustrated in the drawings, the disclosure is not restricted to the above embodiments. Each of the components of the puncture apparatus can be replaced by a component having such a configuration as to be able to exhibit an equivalent function. Besides, the puncture apparatus may have an arbitrary structure or structures added thereto, and the puncturing method may have an arbitrary step or steps added thereto.

In addition, the puncture apparatus and puncturing method according to the present disclosure may be a combination of arbitrary two or more of the configurations (characteristic features) adopted in the aforementioned embodiments.

Besides, while the needle body is detachably held on the main body of the sheath in the fifth embodiment above, this configuration is not restrictive. For instance, a configuration in which the needle body is fixed to the main body, such as a configuration wherein the main body and the needle body are integrally formed, may also be adopted. In that case, after a living body is punctured by the puncture member and the needle body is made to protrude to the outside of the living body, the needle body may be cut by use of a pair of scissors or the like, whereby the distal end opening of the main body can be opened.

While a configuration in which the main body of the sheath can be separated into a distal split piece and a proximal split piece has been described in the fifth exemplary embodiment above, this main body configuration is not restrictive. A configuration in which a distal end portion and a proximal end portion of the main body are not separable from each other may also be adopted. Specifically, the main body may be configured to be a single tube. In that case, the aforementioned state-maintaining mechanism is also omitted.

In addition, while a configuration in which the implant main body is inserted into the puncture member after the puncture member is placed in the inside of a living body has been described in the above embodiments, this configuration is not restrictive. The implant main body may be accommodated in the puncture member, from the beginning. In that case, it can be preferable, for example, for the implant main body to be set fixed relative to the needle tip.

In accordance with an exemplary embodiment, the puncture apparatus of the present disclosure is applied to an apparatus to be used in embedding in a living body an embeddable implant for treatment of female urinary incontinence has been disclosed in the above exemplary embodiments, this is not restrictive of the use of the puncture apparatus.

Examples of the object to which the present disclosure is applicable include pelvic floor diseases inclusive of excretory disorders (urinary urgency, frequent urination, urinary incontinence, fecal incontinence, urinary retention, dysuria, etc.), pelvic organ prolapse, vesicovaginal fistula, urethrovaginal fistula, and pelvic pain, which would be attendant on weakening of the group of pelvic floor muscles. The pelvic organ prolapse include such diseases as cystocele, enterocele, rectocele, and hysterocele, or such diseases as anterior vaginal prolapse, posterior vaginal prolapse, vaginal apical prolapse, and vaginal vault prolapse, which are denominations based on classification of the vaginal wall part being prolapsed.

In addition, examples of overactive tissue include the bladder, vagina, uterus, and bowels. On the other hand, examples of lessactive tissue include bones, muscles, fascias, and ligaments. Especially in relation to the pelvic floor diseases, examples of the lessactive tissue include obturator fascia, coccygeus fascia, cardinal ligament, uterosacral ligament, and sacrospinous ligament.

Examples of the procedure interlocking an overactive tissue in the pelvic floor disorder with the lessactive tissue, there are included a retropubi sling surgery, a transobturator sling surgery (Transobturator Sling Surgery; Transobturator Tape: TOT), a tension-free vaginal mesh (Tension-free Vaginal Mesh: TVM) surgery, a uterosacral ligament suspension (Uterosacral Ligament Suspension: USLS) surgery, an iliococcygeus fascia fixation surgery, and a coccygeus fascia fixation surgery.

The detailed description above describes puncture apparatus and puncturing method. The disclosure is not limited, however, to the precise embodiments and variations described. Various changes, modifications and equivalents can effected by one skilled in the art without departing from the spirit and scope of the disclosure as defined in the accompanying claims. It is expressly intended that all such changes, modifications and equivalents which fall within the scope of the claims are embraced by the claims.

What is claimed is:

1. A puncture apparatus comprising:
a puncture needle which punctures a biological tissue, the puncture needle being elongated and able to be curved; and
a curvature-changing unit which changes curvature of the puncture needle in a process of puncturing the biological tissue by the puncture needle.

2. The puncture apparatus according to claim 1,
wherein the curvature-changing unit is configured to cause the puncture needle to sequentially perform a first puncture and a second puncture, and wherein a curvature of the puncture needle during the second puncture is greater than a curvature of the puncture needle during the first puncture.

3. The puncture apparatus according to claim 2,
wherein the puncture apparatus forms a puncture hole in the biological tissue prior to a placement of an implant placed indwelling between a urethral lumen and a vaginal lumen, and
the first puncture is performed during when a needle tip of the puncture needle is moved from a living body surface to a point of immediately before reaching an obturator foramen on one side, and the second puncture is performed during when the needle tip of the puncture needle is moved from the point of immediately before reaching the obturator foramen on the one side to cross a region between the urethral lumen and the vaginal lumen and is moved past an obturator foramen on other side.

4. The puncture apparatus according to claim 2, further comprising:
a restriction unit which performs the first puncture;
a curved portion which performs the second puncture, the curved portion configured to be insertable in the restriction unit; and
wherein the restriction unit restricts curvature of the curved portion.

5. The puncture apparatus according to claim 4,
wherein the curved portion is curved in an arcuate shape in a natural state.

6. The puncture apparatus according to claim 4, comprising:
an operating unit which deforms the curved portion into an arcuate state.

7. The puncture apparatus according to claim 4,
wherein the curved portion is configured that rotation of the curved portion about an axis along a direction of puncture is restricted by insertion of the curved portion in the restriction unit.

8. The puncture apparatus according to claim 1,
wherein the puncture needle is composed of a plurality of interlocking pieces, a linear portion extending from a proximal end of the plurality of interlocking pieces, and a needle tip on a distal end of the plurality of interlocking pieces.

9. The puncture apparatus according to claim 8, comprising:
a lock mechanism configured to maintain a curved state of the puncture needle, the lock mechanism including a helical compression spring disposed in a compressed state between a flange portion provided at a proximal portion of the puncture needle and a flange portion provided at a proximal portion of an operating wire.

10. The puncture apparatus according to claim 1,
wherein the puncture needle is composed essentially of a wound portion having a band-shaped body wound spirally, and a linear portion extending proximally from the wound portion.

11. The puncture apparatus according to claim 2,
wherein curvature of the first puncture is substantially zero.

12. A method of puncturing a biological tissue to form the biological tissue with a bow-like puncture hole, the method comprising:
puncturing the biological tissue in such a manner that curvature of the puncture hole varies along a longitudinal direction of the puncture hole.

13. The method according to claim 12, comprising:
puncturing the biological tissue with puncture needle, the puncture needle being elongated and able to be curved; and
changing a curvature of the puncture needle with a curvature-changing unit which changes the curvature of the puncture needle in a process of puncturing the biological tissue.

14. The method according to claim 13, comprising:
causing the puncture needle to sequentially perform a first puncture and a second puncture, and wherein a curvature of the puncture needle of the second puncture is greater than a curvature of the puncture needle of the first puncture.

15. The method according to claim 14, comprising:
placing an implant placed indwelling between a urethral lumen and a vaginal lumen after the process of puncturing the biological tissue.

16. The method according to claim 15, comprising:
performing the first puncture when a needle tip of the puncture needle is moved from a living body surface to a point of immediately before reaching an obturator foramen on one side; and
performing the second puncture when the needle tip of the puncture needle is moved from the point of immediately before reaching the obturator foramen on the one side to cross a region between the urethral lumen and the vaginal lumen and is moved past an obturator foramen on other side.

17. The method according to claim 13, comprising:
performing the first puncture with a restriction unit;
performing the second puncture with a curved portion, the curved portion configured to be insertable in the restriction unit; and
restricting curvature of the curved portion with the restriction unit.

18. The method according to claim 17,
wherein the curved portion is curved in an arcuate shape in a natural state.

19. The method according to claim 18, comprising:
deforming the curved portion into an arcuate state with an operating unit.

20. The method according to claim 18, comprising:
restricting rotation of the curved portion about an axis along a direction of puncture by insertion of the curved portion in the restriction unit.
